(19) United States
(12) Patent Application Publication
(10) Pub. No.: US 2026/0241133 A1
(43) Pub. Date: Aug. 20, 2026

(54) TUBE WINDERS FOR RESPIRATORY ASSISTANCE SYSTEMS

(71) Applicant: Fisher & Paykel Healthcare Limited, Auckland (NZ)

(72) Inventors: James William Francis ELLERY, Auckland (NZ); Rebecca Ellen GUMBLEY, Auckland (NZ); Maximiliano PLODER, Auckland (NZ); Samuel Graham BOGGS, Auckland (NZ); Jongsung Danny JUN, Auckland (NZ)

(21) Appl. No.: 19/165,451

(22) PCT Filed: Mar. 15, 2024
(86) PCT No.: PCT/IB2024/052513

§ 371 Date: Sep. 15, 2025

Related U.S. Application Data
(60) Provisional application No. 63/490,737, filed on Mar. 16, 2023.

(51) Int. Cl. A61M 16/10; A61M 16/00

(52) U.S. Cl. CPC A61M 16/109; A61M 16/0003; A61M 2205/0216; A61M 2205/3379; A61M 2209/00

(57) ABSTRACT

A tube winder, tube winder assembly and a humidification assembly for a respiratory assistance system. In some cases, the tube winder has body configured to connect to a single port of the humidification chamber, and an outer side wall substantially surrounding the single port, an outer surface of the outer side wall configured to support a wound tube. In some cases, the body, when connected to the port, is configured to extend away from the port in a direction away from the connection direction between the humidification chamber and a humidification base. In some cases, the tube winder is configured to remain attached to the humidification chamber during connection of the humidification chamber to the humidification base. In some cases, the body has a cover configured to at least partially occlude the opening of the port when the tube winder is connected to the port, the cover having an aperture configured to allow a retainer to pass into the port.

### BACKGROUND

### Technical Field

The present disclosure generally relates to respiratory assistance systems. Particular aspects of the present disclosure relate to tube winders for respiratory assistance systems, in particular a tube winder for a tube to be located on respiratory assistance systems. Other aspects relate to respiratory assistance systems having features for improved assembly and/or usability.

### Description of the Related Art

A respiratory assistance system may be used to provide respiratory gases to a patient from a gases source via an inspiratory conduit in fluid communication between the gases source and a patient interface. Examples of a patient interface may include an oral mask, a nasal mask, a full face mask covering both the nares and mouth of a patient, a nasal cannula, a tracheal mask, or an endotracheal tube. In a respiratory assistance system where the gases source is a ventilator, gases exhaled by the patient into the patient interface may be returned via an expiratory conduit in fluid communication between the patient interface and the ventilator. The inspiratory conduit and the expiratory conduit may be connected to the patient interface via a wye-piece connector.

A respiratory assistance system may include a humidification device to condition respiratory gases provided to the patient. The humidification device may include a humidification chamber containing liquid and a heater adjacent the humidification chamber to heat the liquid to produce vapor. The humidification device may be positioned in the fluid communication path between the gases source and the patient interface to heat and/or humidify respiratory gases prior to delivery via the inspiratory conduit to the patient interface. The humidification chamber can include a mechanism such as float to help reduce the likelihood of the level of liquid in the chamber exceeding a particular level. The float may rise and fall with the level of liquid in the chamber. When the liquid reaches a certain level, the float may cause actuation of a mechanism, such as a valve, to obstruct or block the port that is connected to the liquid conduit to stop or slow further ingress of liquid into the chamber. The chamber can include an opening or port for the connection of a liquid tube such as a feedset tube. The liquid tube can extend from the chamber and may connect to a spike for a water bag.

A port cap assembly may be used with the humidification chamber, for example during shipping and storage. The port cap can cover the inlet port. The post of the port cap can include an extension configured to be received inside of the chamber to secure in position the float inside the chamber. The port cap assembly can include a lifting structure. The lifting structure may be pivotable relative to the port cap and may comprise a ring, tab, hook, or any other suitable structure. The lifting structure serves as a visual indicator to help encourage removal of the port cap while also facilitating the removal of the port cap. The port cap may have a space in which a wound tube can be stored.

### SUMMARY

A humidification device for use in a respiratory assistance system may have a tube winder including one or more components configured to mate with one or more corresponding components of the humidification device. Such a tube winder may allow the tube winder to stay in position while the humidification device is installed and may alternatively or also support, guide, or orientate a retainer and/or a wound tube.

The humidification device may include a humidification chamber. The humidification chamber may include an inlet port for receiving relatively dry gases and an outlet port for delivering relatively moist or humid gases to a patient. The chamber can further include an opening or port for the connection of a liquid tube. The liquid tube can extend from the chamber and may connect to a spike for a water bag. The liquid tube can be integrally formed with or permanently coupled to the chamber. The spike can be coupled to the liquid tube via an adhesive, sonic welding, an interference fit, or any other suitable means. The spike may include a vent. If the spike is inserted into, for example, a plastic, collapsible bag, the vent may be plugged. However, if the spike is inserted into a rigid container, such as a glass bottle, the vent may be open to allow air to enter the container to help reduce or prevent negative pressures in the container. The vent can include a filter that is permeable to gases but impermeable to liquids.

In use, the liquid tube conveys a liquid, for example, water, from a liquid source, such as a water bag, saline bag, or the like, to the chamber. The heater adjacent the chamber, such as a heater plate engaged with the base of the chamber, heats the chamber, and causes at least some of the chamber contents to evaporate. The chamber can include features to help reduce the likelihood of the level of liquid in the chamber exceeding a particular level. For example, the chamber can include one or more floats. Each of the floats may rise and fall with the level of liquid in the chamber. When the liquid reaches a certain level, each of the floats may cause actuation of a mechanism to obstruct or block the port that is connected to the liquid tube to stop or slow further ingress of liquid into the chamber. The mechanism may be a valve. Other similar features also can be used. A plurality of floats may be used, each float adapted to stop the further ingress of liquid into the chamber. To this end, a second float may provide a backup or safety mechanism in case the first float fails, thereby further reducing the likelihood of the chamber overfilling.

US20170252531A1, incorporated herein by reference, discloses various features for improving assembly and/or usability of humidification systems, including an improved cap for a port of a humidification chamber. In the arrangements described in US20170252531A1, the inlet port extends generally vertically, while the outlet port extends generally horizontally, in an elbow configuration extending from the chamber generally vertically and then generally horizontally, or in some orientation other than generally vertically. A port cap may be used to cover the inlet port during shipping and storage, for example. The port cap can include a collar and a post with the collar having a port encircling the support. The support may be a generally planar surface that includes an opening that is sized and configured to receive the inlet port, but other configurations are possible.

US20170252531A1 states that the post can include a lid. The lid can include a downwardly extending flange. The lid and the flange may generally enclose the wrapped liquid tube. A spike can be connected to the liquid tube. The spike can be received within a sleeve. The sleeve may be connected to the port cap, more particularly, to at least one of the collar and the post. The sleeve can be joined to, or integrally formed with, the retainer and may be pivotally connected to the collar or the retainer. The sleeve may have a flange that is connected to the retainer that extends upward from an edge of the retainer. In such configurations, the sleeve can pivot downwardly when the spike is positioned within the sleeve.

Additionally, the port cap can include a lifting structure. The lifting structure may be pivotable relative to the port cap and may comprise a ring, tab, hook, or any other suitable structure. The lifting structure serves as a visual indicator to help encourage removal of the port cap while also facilitating the removal.

In some cases it is an object of the present invention to provide a tube winder to assist a user in the set-up of a humidification chamber and/or in the storage of a tube on the tube winder or at least provide a useful alternative.

In one aspect the present invention may broadly be said to comprise a tube winder for a humidification chamber comprising at least one port, the tube winder comprising: a body configured to connect to a single port of the humidification chamber, the body comprising an outer side wall substantially surrounding the single port, an outer surface of the outer side wall configured to support a wound tube.

In some cases the body comprises an engagement surface configured to engage the single port of the humidification chamber.

In some cases at least a portion of the engagement surface extends away from the outer side wall.

In some cases at least a portion of the engagement surface comprises at least a portion of an inner surface of the outer side wall.

In some cases the body comprises an inner side wall, at least a portion of the engagement surface being on at least a portion of the inner side wall.

In some cases the engagement surface is configured to substantially surround the single port.

In some cases the engagement surface has a guide configured to orientate the tube winder on the outlet.

In some cases the guide comprises one or more protrusions and/or recesses configured to mate with corresponding recesses and/or protrusions on the single port to secure and/or orientate the tube winder when the tube winder is connected and/or connecting to the single port.

In some cases the guide is resiliently deformable so as the tube winder releasably secures to the port.

In some cases the guide comprises a slot extending through the engagement surface and/or the side wall.

In some cases the mouth of the slot is wider than a body of the slot, so as to guide the tube winder into a correct orientation.

In some cases the slot comprises a catch to secure the tube winder on the port.

In some cases the body comprises a cover, the cover configured to at least partially occlude the single port when the tube winder is connected to the single port.

In some cases the cover comprises an aperture configured to be located above the opening of the port when the tube winder is connected to the port, the aperture configured to allow a retainer to pass therethrough.

In some cases the aperture is configured to locate the retainer apart from a component within the single port when the retainer is located in the aperture and the tube winder is engaged to the single port.

In some cases the tube winder body comprises an upper frame and/or a lower frame, wherein the outer side wall extends from or between the upper and/or lower frame.

In some cases the side wall comprises a plurality of members extending from or between the upper and/or lower frame.

In some cases the outer side wall comprises an upper and/or a lower flange extending therefrom, the upper and/or lower flange configured to retain the tube on the outer side wall.

In some cases the outer side wall is discontinuous and/or comprises recesses and/or comprises protrusions.

In some cases the humidification chamber comprises a second port, wherein the tube winder does not surround and/or contact the second port.

In some cases the body is elongate.

In some cases the body and/or the outer side wall extends in a first dimension substantially the width of the single outlet and extends in a second dimension a length greater than the width of the single outlet.

In some cases the outer side wall is substantially rectangular.

In some cases the outer side wall comprises curved or chamfered corners.

In some cases at least a part of at least one an end of the outer side wall is flat.

In some cases the outer side wall is configured to retain the wound tube.

In some cases the outer side wall comprises at least one tube retainer for retaining the wound tube against the outer side wall.

In some cases the tube retainer comprises at least one protrusion from the outer side wall and/or at least one recess or opening in the outer side wall.

In some cases comprising a holder configured to support an end of the tube.

In some cases the holder extends from, or is connected to, the tube winder body.

In some cases the holder is configured to support a spike attached to the end of the tube.

In some cases the holder comprises a sleeve configured to hold the tube and/or spike.

In one aspect the present invention may broadly be said to comprise a tube winder assembly comprising: a tube winder as in any other aspect; and a retainer comprising a leg configured to pass through the tube winder when connected to the single port.

some cases the leg is elongate and comprises at least one engagement portion configured to engage a surface within the humidification chamber.

In some cases the engagement portion is at or near an end of the leg.

In some cases the surface is a surface of a float and/or float assembly within the humidification chamber.

In some cases the tube winder comprises an aperture, the aperture configured to align the retainer relative to the aperture and/or the port.

In some cases the retainer comprises an aligner and/or an alignment surface configured to align the retainer relative to the aperture and/or the port.

In some cases the aligner comprises a shape of the retainer and a corresponding shape of the aperture and/or the port.

In some cases the aligner comprises a shape of the leg.

In some cases the aligner comprises at least one edge of the leg and/or an upper surface of the retainer.

In some cases the aperture and/or the aligner are configured and/or shaped to allow the retainer to be inserted into the tube winder in a single alignment.

In some cases the retainer comprises a tube retainer. In some cases the tube retainer is configured to retain the wound tube to the tube winder when the retainer is engaged with and/or located in the tube winder.

In some cases the tube retainer comprises an extension portion configured to extend across an upper surface of the tube winder and a retention portion configured to retain the wound tube.

In some cases the retention portion retains one, or at least one, layer of the wound tube. In some cases the extension portion extends from an end of the upper surface of the retainer. In some cases the retention portion is configured to release the wound tube when the retainer is removed from the tube winder.

In some cases comprising a tube, the tube configured to be wound about the tube winder.

In some cases the tube comprises a first end connected or connectable to the humidification chamber and/or comprises a second end connected or connectable to a spike.

In some cases the tube is a liquid tube.

In some cases the body comprises an inner wall configured to engage the inside of the single port.

In some cases the inner wall comprises a guide portion configured to guide the retainer into the port.

In some cases the guide portion is adjacent the aperture.

In some cases the inner wall comprises one or more protrusions to engage corresponding protrusions on the retainer.

In some cases the inner wall comprises a protrusion at each end of the guide portion.

In some cases the protrusions are shorter than the guide portion.

In some cases the corresponding protrusion on the retainer engages beneath the protrusions.

In some cases the guide portion has a smaller outside radius than the protrusions.

In some cases the inner wall forms a substantially circular wall configured to fit within the port.

In one aspect the present invention may broadly be said to comprise a humidification assembly comprising a tube winder as described in the other aspects or cases and a humidification chamber, the humidification chamber comprising at least one port.

In one aspect the present invention may broadly be said to comprise a humidification assembly comprising a tube winder and a humidification chamber, the humidification chamber comprising: at least one port, the tube winder comprising: a body configured to connect to a single port of the humidification chamber, the body comprising an outer side wall substantially surrounding the single port, an outer surface of the outer side wall configured to support a wound tube.

In some cases comprising a tube connected to the humidification chamber, the tube configured to be wound about the tube winder.

In some cases comprising a humidification base, the humidification chamber configured to connect to the humidification base.

In some cases the humidification base comprises a cartridge configured to connected to the humidification chamber.

In some cases the humidification base and/or cartridge has one or more probes and/or sensors extending therefrom, at least one of the one or more probes and/or sensors configured to engage the at least one port of the humidification chamber.

In some cases at least one of the one or more probes or sensors is configured to extend into a port of the humidification chamber.

In some cases at least one of the ports comprises one or more grommets configured to cover an opening into the port to protect an associated probe and/or sensor and/or engage with an associated probe and/or sensor.

In some cases the grommets extend within the at least one port.

In some cases at least one of the ports comprises one or more protrusions on the insider surface of the port, the protrusions configured to protect the one or more grommets. The protrusions may be referred to as protective protrusions.

In some cases the protrusions are axial protrusions.

In some cases the protrusions are axial ribs.

In some cases there are two protrusions, one protrusion located on each side of the grommet.

In some cases the protrusions are configured to abut an edge of a retainer positioned in the port.

In some cases the protrusions are extend from above the grommet into the port.

In some cases the protrusions extend towards the outlet of the port.

In some cases the grommets are positioned on a portion of the port wall protruding towards the center of the port.

In some cases the protrusions are positioned on the protruding portion of the port wall.

In some cases the protrusions have a constant cross section.

In some cases the protrusions widen below the one or more grommets.

In some cases the protrusions are substantially straight.

In some cases at least one of the ports is an inlet port and/or and outlet port.

In some cases the humidification base and/or the cartridge is configured to engage with an upper surface of the humidification chamber to align the humidification chamber with the humidification base.

In some cases the humidification base and/or the cartridge has a protrusion configured to engage the upper surface.

In some cases the humidification base and the humidification chamber are configured to have one or more electrical, mechanical, and/or pneumatic connections therebetween.

In some cases one of the one or more electrical, mechanical and/or pneumatic connections is provided as part of a connector to an outlet of the humidification chamber.

In some cases the humidification chamber comprises one or more components within the chamber, the components being configured to be retained.

In some cases one of the one or more components comprises a float configured to control the water level in the humidification chamber in use.

In one aspect the present invention may broadly be said to comprise a tube winder, for a humidification chamber comprising at least one port and having a back side configured to mate with a humidification base, the tube winder comprising: a body configured to connect to a port of the humidification chamber, the body having an elongate surface configured to support a wound tube, wherein the tube winder is configured so that the longitudinal axis of the elongate surface extends away from the port towards the front of the humidification chamber, when on the port.

In one aspect the present invention may broadly be said to comprise a tube winder, for a humidification chamber comprising at least one port and having a back side configured to mate with a humidification base, the tube winder comprising: a body configured to connect to a port of the humidification chamber the body comprising: an outer side wall surrounding the port, the outer side wall comprising an outer surface configured to support a wound tube, and wherein the tube winder is configured extend away from the port towards the front of the humidification chamber, in use.

In one aspect the present invention may broadly be said to comprise a tube winder, for a humidification chamber comprising at least one port, the tube winder comprising: a body configured to connect to a port of the humidification chamber the body comprising: an outer side wall surrounding the port, the outer side wall comprising an outer surface configured to support a wound tube, and wherein, when connected to the port, the outer surface of the tube winder is configured or configurable to extend away from the port in a selected direction.

In one aspect the present invention may broadly be said to comprise a tube winder, for a humidification chamber comprising at least one port and configured to connect to a humidifier base on a connection side, the tube winder comprising: a body configured to connect to a port of the humidification chamber the body comprising: an outer side wall surrounding the single port, the outer side wall comprising an outer surface configured to support a wound tube, wherein the body, when connected to the port, is configured to extend away from the connection side.

In one aspect the present invention may broadly be said to comprise a tube winder, for a humidification chamber comprising at least one port and configured to connect to a humidifier base in a first direction, the tube winder comprising: a body configured to connect to a port of the humidification chamber the body comprising: an outer side wall surrounding a port, the outer side wall comprising an outer surface configured to support a wound tube, wherein the body, when connected to the port, is configured to extend away from the port in a second direction, the second direction being different to the first direction.

In some cases the body is configured to only extend away from the port in a second direction.

In some cases the outer side wall surrounds a single port of the humidification chamber.

In some cases the humidification chamber is connectable to the humidification base while the tube winder is connected to the humidification chamber.

In some cases the first direction is substantially opposite the second direction.

In some cases the second direction is towards the front of the humidification chamber.

In some cases the second direction is substantially parallel to a direction of a second port of the humidification chamber.

In some cases comprising an orientator configured to orientate the body relative to the port to control the second direction relative to the first direction.

In some cases the orientator is configured to orientate the body on the port towards the second direction.

In some cases the orientator comprises a protrusion or recess on an engagement surface of the body.

In some cases the orientator comprises a catch configured to engage with the port in the second direction.

In some cases the orientator is part of, or on, the outer side wall of the body.

In some cases the orientator comprises a slot on the tube winder body.

In some cases comprising a cover to at least partially occlude the port.

In some cases the tube winder is as described in any of the previous aspects or cases.

In one aspect the present invention may broadly be said to comprise a humidification assembly comprising: a tube winder configured to support a wound tube, and a humidification chamber configured to connect to a humidification base, the humidification chamber comprising at least one port, the tube winder connected to a port of the humidification chamber, wherein the tube winder is configured to remain connected to the humidification chamber while the humidification chamber is connected to the humidification base.

In some cases comprising a tube connected to the humidification chamber, the tube configured to be wound about the tube winder.

In some cases comprising the humidification base.

In some cases the humidification base comprises a cartridge configured to connected to the humidification chamber.

In some cases the humidification base and/or cartridge comprises one or more probes and/or sensors extending from the base and/or cartridge, at least one of the one or more probes and/or sensors configured to engage the at least one port of the humidification chamber on which the tube winder is attached.

In some cases at least one of the one or more probes and/or sensors is configured to extend into one of the at least one ports of the humidification chamber.

In some cases at least one of the at least one ports has one or more grommets configured to surround an opening into the port and/or engage with an associated probe and/or sensor.

In some cases at least one of the grommets extend within the at least one port.

In some cases at least one of the ports is an inlet and/or and outlet port.

In some cases the tube winder is configured to connect to the port above the one or more grommet.

In some cases at least one of the ports comprises one or more protrusions on the insider surface of the port, the protrusions configured to protect the one or more grommets.

In some cases the protrusions are axial protrusions.

In some cases the protrusions are axial ribs.

In some cases there are two protrusions, one protrusion located on each side of the grommet.

In some cases the protrusions are configured to abut an edge of a retainer positioned in the port.

In some cases the protrusions are extend from above the grommet into the port.

In some cases the protrusions extend towards the outlet of the port.

In some cases the grommets are positioned on a portion of the port wall protruding towards the center of the port.

In some cases the protrusions are positioned on the protruding portion of the port wall.

In some cases the protrusions have a constant cross section.

In some cases the protrusions widen below the one or more grommets.

In some cases the protrusions are substantially straight.

In some cases the humidification base and/or the cartridge is configured to engage with an upper surface of the humidification chamber to align the humidification chamber with the humidification base.

In some cases the humidification base and/or the cartridge has a protrusion configured to engage the upper surface.

In some cases the humidification base and the humidification chamber are configured to have one or more electrical, mechanical and/or pneumatic connections therebetween.

In some cases one of the one or more electrical and/or pneumatic connections is provided as part of a connector to an outlet port of the humidification chamber.

In some cases the humidification chamber comprises one or more components within the chamber, the components being configured to be retained.

In some cases one of the one or more components comprise a float configured to control the water level in the humidification chamber in use.

In some cases further comprising a retainer configured to extend through the tube winder into the port and/or the humidification outlet.

In some cases the retainer is configured to be aligned by the tube winder and/or the port so as to avoid contact with components within the humidification chamber and/or the port.

In some cases the retainer is aligned to avoid contact with a grommet in the port.

In some cases the tube winder comprises an inner wall configured to engage the inside of the single port.

In some cases the inner wall comprises a guide portion configured to guide the retainer into the port.

In some cases the guide portion is adjacent an aperture through the cover of the tube winder.

In some cases the inner wall comprises one or more protrusions to engage corresponding protrusions on the retainer.

In some cases the inner wall comprises a protrusion at each end of the guide portion.

In some cases the protrusions are shorter than the guide portion.

In some cases the corresponding protrusion on the retainer engages beneath the protrusions.

In some cases the guide portion has a smaller outside radius than the protrusions.

In some cases the inner wall forms a substantially circular wall configured to fit within the port.

In some cases the tube winder comprises an outer side wall with an outer surface configured to support the wound tube.

In some cases the tube winder is a tube winder as described in the other aspects or cases.

In some cases the body is configured to connect to a single port of the humidification chamber.

In one aspect the present invention may broadly be said to comprise a tube winder, for a humidification chamber comprising at least one port, the humidification chamber configured to connect to a humidifier base in a first direction, the tube winder comprising: a body configured to connect to a port of the humidification chamber, wherein the body comprises a cover configured to at least partially occlude the opening of the port when the tube winder is connected to the port, the cover comprising an aperture configured to allow a retainer to pass therethrough into the port.

In some cases comprising an orientator configured to orientate the body relative to the port on the humidification chamber.

In some cases the orientator comprises a slot.

In some cases the aperture is configured to locate a leg of the retainer relative to the port and/or the tube winder.

In some cases the aperture is shaped to position and/or guide the leg of the retainer relative to the port and/or the tube winder.

In some cases the aperture is configured to secure the retainer in a selected position.

In some cases the aperture comprises a wall configured to guide the retainer.

In some cases the wall extends along at least a portion the circumference of the aperture.

In some cases the wall extends below the cover of the tube winder.

In some cases the body is configured to connect to a single port of the humidification chamber.

In some cases the cover comprises a closure configured to close the aperture when the retainer is not present.

In some cases the cover and the retainer are configured to fully occlude the opening when the tube winder assembly is connected to the port.

In some cases the body comprises an outer side wall substantially surrounding the single port, an outer surface of the outer side wall configured to support a wound tube.

In some cases the outer side wall extends around the cover.

In some cases body has an engagement surface configured to engage the single port of the humidification chamber.

In some cases the body comprises an inner wall configured to engage the inside of the single port.

In some cases the inner wall comprises a guide portion configured to guide the retainer into the port.

In some cases the tube winder is as described in the other aspects or cases.

In one aspect the present invention may broadly be said to comprise a tube winder assembly comprising: the tube winder as described in the other aspects or cases and a retainer, wherein the retainer is configured to engage a surface and/or component inside the humidification chamber.

In some cases the cover and the retainer are configured to fully occlude the opening when the tube winder assembly is connected to the port.

In some cases the tube winder comprises an inner wall configured to engage one or more protrusions on the retainer.

In some cases, the tube winder comprises a body with an outer side wall substantially surrounding a single port of the humidification chamber, wherein a tube can be wound about and supported by the outer side wall. Storing the wound tube on a single port means that the remainder of the chamber is unencumbered and can be connected easily to a humidification base while the tube winder is attached. Winding the tube around the outer surface of an outer side wall surrounding the single port means that the tube is easily and securely wrapped onto the winder and the winder can be moved and replaced without the tube unravelling, it also makes manufacturing of the tube winder easier as the tube remains compactly stored on the winder, and it is easier to wind the tube onto the tube winder as it is accessible on the outside and does not require any other component(s) to be in position. The tube is also easier for a user to remove when on the external wall than if wound internally or inserted into the tube winder. The tube winder provides a convenient storage location as well as locating the tube on the humidification chamber ready for use. The use of a single port ensures that the other port, and in some cases the area of the humidification chamber between them or a central area of the humidification chamber, is unencumbered by the tube winder and or wound tube. This makes set up easier and allows the tube winder to stay in place for longer during installation by not blocking access to other components. The outer side wall may extend away from the single port in a substantially a single direction to form an elongate side wall and/or tube winder. A liquid tube is described, however any suitable tube associated with the humidification apparatus may be wound on the tube winder.

In some cases, an engagement surface of the tube body is configured to engage the port of the humidification chamber. Advantageously a surface is used to engage with or connect to the port to provide a secure connection. In some cases, the surface substantially surrounds or encircles the single port. In some cases, the surface partially and/or completely surrounds the single port. In some cases, a catch may be used instead of, or together with, the engagement surface. In some cases, the engagement surface is at least a portion of, an inner surface of the side wall. This means that the tube is would about an outer surface of the wall that engages the port on an inner surface, reducing the width and/or overall size of the tube winder. This may provide additional space on the humidification chamber. In some cases, the engagement surface and the side wall may be separate parts of the body, with the side wall being an outer wall surrounding the inner wall, or engagement wall which has the engagement surface thereon. In some cases, the engagement wall overlaps with the outer wall, at least in part. In some cases, the engagement surface and/or engagement wall extends substantially around the port in use. The use of a single wall reduces the amount of material used in the tube winder and simplifies the construction, minimising the space taken up by the tube winder. In some cases a non-overlapping portion occurs where the outer side wall extends away from the port to provide a longer winding surface and/or an elongate tube winder. The inner wall may extend away from the side wall to encircle or substantially surround the port, providing a larger engagement surface. In some cases the body comprises the engagement surface and the outer wall configured to support the tube. In some cases the body is unitary. The body may have additional features attached to it, such as a holder for the tube end.

In some cases the tube winder has a single side wall with the inner surface engaging the single port and the outer surface providing the winding surface. This allows a single walled tube winder to achieved both functions of engagement and supporting the tube, reducing the size and complexity of the winder. In some cases further portions of the outer side wall then extend to surround the port and/or form an elongate extension of the winding surface.

In some cases the engagement surface completely, or almost completely surrounds the single port. In some cases the engagement surface may be discontinuous. For example the engagement surface may have a plurality of ribs extending along the surface or may have one or more recesses. The discontinuities or recesses may allow the engagement surface to fit over features, such as protrusions, on the single port. In some cases the engagement surface engages with the outer surface of the single port.

The tube winder may comprise a guide configured to orientate the tube winder on the port. The guide may restrict the engagement between the tube winder and the port to one, two or a plurality of positions, so as to ensure the tube winder becomes correctly positioned on the port. The guide may have one or more protrusions or recesses configured to mater with corresponding protrusions or recesses on the single port. In some cases the guide may also secure or attach the tube winder to the port. The guide may have a catch which engages the port to limit further relative movement.

In some cases the guide may be formed by a shape or dimension of the engagement surface and/or engagement wall. The guide may snap-fit or otherwise releasably secure to the single port. The tube winder may be configured to connect to the port along the axial axis of the port. In some cases the guide is released by rotating the tube winder relative to the single port. In some cases the rotation is upwards relative to the opening of the single port. In some cases the guide helps to orientate the tube winder when it is being placed or replaced on the single port. In some cases the guide has an opening which is widened, for example by being flared, so as to guide onto a protrusion on the port (or vice-versa). In some cases the guide has a recess above the opening to latch to a protrusion on the port. In some case the connection features on the port are configured to engage with other humidifier components in use, such as conduits connected to the single port. The guide helps to orientate the winder relative to the port when the winder is engaged or being engaged to the port. For example, if the winder is not in the correct orientation the guide, interacting with the port, for example with a protrusion on the port, urges the winder into a correct orientation relative to the port.

For example the guide may comprise a slot through the engagement wall (which may also be the side wall) configured to engage a protrusion on the port. This may mean the tube winder can only be inserted when the slot and the protrusion are aligned. The protrusion may also serve further purposes on the humidification chamber, for example aligning a conduit to the port. The slot may extend from a first edge of the tube winder (for example the bottom of the engagement surface), toward a top surface of the tube winder. The mouth of the slot may be wider than the top or body of the slot so as to urge the tube winder into the correct position. The side walls of the slot may have recesses and/or protrusions to engage with a protrusion on the port. The recesses and/or protrusions may form a catch to secure the position of the tube winder relative to the port. The portions of the wall forming the sides of the slot may be flexible so as to be reversibly deformable. This flexibility allows the sides to widen over the protrusion before narrowing to secure the recesses around the protrusions. The slot may be relatively wide to provide strength to the connection and/or make connection easier. The width may be greater than 10%, greater than 15%, greater than 20% or greater than 25% of the circumference of the tube winder.

A second guide, such as the slot, may be configured on the tube winder. The second guide may be substantially identical to the first guide. The second guide may be on the opposite side to the first guide. The second guide may be configured to increase the flexibility of the sides of the guide to make engagement easier. In some cases the second guide may allow a second position of the tube winder to be engaged and/or provide improved securing of the tube winder.

The tube winder body may have a cover (configured to at least partially occlude or cover the opening of the single port in use. The cover may be a portion of the upper surface of the tube winder. The cover prevents unwanted entry into the single port. This is advantageous where the tube winder can be kept in place during installation, and where the cover is an integral part of the tube winder body, so as it cannot be separately removed.

The cover may have an aperture configured to allow a retainer, such as a leg, or elongate member, to pass therethrough and into the chamber in use. The aperture may be configured to control the orientation of the retainer. This may be configured to prevent the retainer from contacting surfaces or components (such as floats) inside the port and/or chamber. An aperture allows a retainer to pass into the port. In some cases the retainer is elongate so as to reach through the port and retain one or more components or surfaces within the humidification chamber. In some cases the aperture and/or the tube winder body have protrusions, recesses or is otherwise shaped, to locate or correctly position the retainer. In some cases the aperture is shaped to guide the retainer into a suitable position. In some cases the aperture allows the retainer to be positioned in a single position. In some cases the retainer extends through to the humidification chamber, however it may extend only to the port, to retain a feature in the port and, or to contact an extension which retains components in the chamber.

In some cases the aperture may have a closure configured to fully occlude the aperture when the retainer is not present, and therefore allow the port opening to be fully covered when the retainer is not present.

The outer surface of the sidewall may support a wound tube in several ways. In some cases the sidewall may be a continuous wall around which the tube is wound. In some cases the sidewall may comprises an upper and/or a lower frame with members passing from or between the frames. In this case the tube would be supported on the spaced apart members, with sections of the tube not directly resting on the side wall. The upper and/or lower frame may be connected to the engagement surface to form part of the tube winder body. The side wall may be designed to reduce weight and/or provide flexibility of use. In some cases it is not a continuous surface. Instead it may be formed from a plurality of ribs extended horizontally or vertically to support the wound tube. The ribs being supported by the body of the tube winder.

In some cases the side wall and/or tube winder body has one, two or a plurality of retainers for retainer the wound tube on the tube winder. The tube retainers may comprise upper and/or lower flanges. The upper and/or lower flanges may extend outwards from the side wall so as to prevent the tube from sliding off the side wall. The flanges may prevent vertical movement of the tube. The upper and/or lower flanges may extend for a portion of the side wall and/or a majority of the side wall and/or for substantially all the circumference of the side wall. In some cases the side wall may surround an opening. In some cases the opening may be configured to allow a user to hold and/or remove the tube winder.

In some cases the tube is retained by a tube retainer. The tube retainer may comprise one or more clips, recesses and/or protrusions. In some cases two protrusions are used to capture a section of the tube. The two protrusions may be spaced apart substantially the diameter of the tube, or slightly less than the diameter of the tube so as to allow the tube to be pushed between them. In some cases the upper and/or lower flanges and/or the upper and/or lower frame may form one of the protrusions. In some cases the tube retainer may comprise a recess in the side wall. The recess may be sized so as to be substantially the diameter of the tube, or slightly less than the diameter of the tube so as to allow the tube to be into the recess. In some cases one, two or a plurality of tube retainers are used.

In some cases the humidification comprises at least first and second ports, but the tube winder surrounds a single port and does not contact the second port. In some cases it does not surround the second port and/or extend substantially towards the second port. Avoiding contact, or at least not surrounding or encircling the second port enables connections to be made to the second port without removal of the tube winder. In some cases the second port is an outlet port. In some cases the second port comprises an elbow. In some cases the outlet of the elbow extends in a direction substantially parallel to the extension of the tube winder from the single port.

The tube winder may have an elongate body. In some cases the elongate body is formed by the outer side wall or is the outer side wall. In some cases the tube winder has an outer side wall configured to support a tube and defining the elongate body. The elongate body may extend in a first direction further than in a second direction. The extension in the first direction may be at least percent, at least 100%, at least 150% or at least 200% greater than the second direction. An elongate winder allows more of the wound tube to extend in a single direction, narrowing the overall profile of the winder. This means that the winder can be advantageously arranged on the humidification chamber to reduce contact or interference with other components. In some cases the first dimension extends substantially the same length as the single port (i.e. enough to surround the single port with a single layer of tube). This minimises this dimension (i.e. the width of the tube winder), while the second dimension can extend away from the single port in the chosen direction. This reduces the bulk of the tube winder around the port, while providing sufficient space for the wound tube. The elongate axis can then be configured to extend in a chosen direction where it may be less likely to contact parts of the humidification chamber or humidification base, in use. The shape of the winder body can be configured to improve usability by rounding the corners. In some cases flat surfaces or edges of the side wall and/or winder body may be used for positioning and/or holding and/or to make it easier to wind the tube on the side wall.

The tube winder may have a holder configured to support the end of the tube. The holder may be attached or attachable to the body of the tube winder. The holder may be attached at a top surface of the tube winder. The holder may be attached by a hinge to allow relative movement of the holder. The holder may be a sleeve to allow the end of the tube to be placed within the holder. The holder may be configured to receive an end connector, such as a water spike, attached to the end of the tube. The holder provides a convenient support for the end of the tube that is wound about the tube winder.

In some cases a tube assembly is provided comprising a tube winder and the retainer with a leg configured to pass through the tube winder. The leg may be configured to pass through the aperture in the cover of the tube winder. The leg may have one or more or a plurality of engagement portions configured to engage one or more surfaces within the humidification chamber. At least one engagement portion may be located at or near a distal end or tip of the leg. This may allow the retainer to secure a float to the bottom of the humidification chamber. The retainer may secure to a float, or any other component in the humidification chamber. Securing to a float may require the retainer to be removed before water is added to the humidification chamber. Advantageously because the retainer must be removed before or with the tube winder the cover of the tube winder may prevent the water being added to the humidification chamber before the retainer has been removed. The aperture of the tube winder may be configured to orientate the retainer, the orientation may be between the leg of the retainer and the edges of the aperture and/or between a top surface of the retainer and the aperture. The retainer may also have an aligner to align itself relative to the port. For example it may have a protrusion or recess, such as a groove, or an edge to align with a corresponding feature in the port.

In some cases the tube winder is configured to have an elongate body that extends away from the connection side of the humidification chamber. The elongate body may extend substantially only in a single direction. That is there may be minimal extension in any other direction, except for required engagement to the port and width of the wound tube. In some cases the extension may be in a second or selected direction which is different to a first direction in which the humidification chamber is configured to connect to the humidification base. The second direction and the first direction may be separated by at least 45 degrees, at least 90 degrees, between 90 and 180 degrees, or may be substantially opposite one another. In some cases the second direction may be towards a front of the humidification chamber and/or away from the back of a humidification chamber. The second direction may be away from an aperture on the port, in some cases the aperture is for one or more sensors. The one or more sensors may be mounted on one or more probes. These arrangements may allow the tube winder to remain on the humidification chamber while it is connected to a base, or while electrical, mechanical, and/or pneumatic connections are made to it. Advantageously this reduces the steps required to set up the device and/or ensures the port is occluded for longer. These features may also mean that the tube winder can be removed at a convenient time after the humidifier chamber has been set-up. These arrangements can also reduce and/or avoid contact between the tube winder and various protrusions, electrical connections, probes, probe mounts and grommets that are typically located on or between the humidification chamber and the humidification base.

In some cases the tube winder is configured to avoid any connections between the humidification chamber and the humidification base. For example, the elongate shape of the tube winder (or at least the side wall) may be configured to extend away from the connections between the humidification chamber and base. Other components on the tube winder, such as the tube holder, may also be configured to extend away from the connections. In some cases the central portion, or a raised portion, of the humidification chamber is also avoided so as to avoid interfering with alignment features, connections or protrusions, in this area. Advantageously, extending in substantially a single direction limits any undesirable interference. The humidification base may include the spine, or other vertical extensions, and/or the cartridge when attached or attachable to the base.

In some cases the tube winder extends in the opposite direction to the back and/or connection side of the humidification chamber. Extending in the opposite direction means that the tube winder at least partially extends away from the connection of the humidifier to the humidification base. This may be exactly opposite (i.e. 180 degrees apart), or at least 90 degrees apart, so that it extends in the reverse direction.

In some cases an orientator on the tube winder is configured to orientate the tube winder so as to force or urge it to be positioned in the second direction, or one or more selected directions. The orientator may be similar to those described above comprising a slot, a protrusion and/or a recess. A catch may be used to secure the tube winder in position. In some cases the tube winder may not extend over a central axis of the humidification chamber. In some cases the tube winder may be contained above one half of the humidification chamber. This avoids additional bulk, such as extensions from the tube winder, getting in the way of the connection and/or orientation devices in the middle of the humidification chamber. The orientator may be in various configurations on the tube winder. For example it may be arranged on a wall of the body. The wall may be the side wall on which the tube is wound (which may be a single wall providing an inner engagement surface and an outer surface for the wound tube). In some cases it may be on an inner wall separate from the outer side wall. In some examples the orientator is the slot describe herein.

The tube winder may have a body to connect to the humidification surface with a cover configured to at least partially occlude the opening of the port when in use, where an aperture in the cover allows the leg of the retainer to pass into the port. Incorporating a cover into the tube winder body ensures that, even once the retainer is removed the tube winder substantially blocks access to the port and prevents entry or contamination of the port during set up and installation. The aperture allows the leg of the retainer to be removed while the cover remains in place. This allows the leg of the retainer to be removed (for instance unlocking floats to allow water into the chamber) without full dismantling of the port. This prevents a user form improperly reinserting the retainer to try and occlude the port opening.

The tube winder can be used to guide the elongate retainer into a position or location relative the port and/or the tube winder when the tube winder is engaged to the port and the retainer positioned in the aperture. The position may properly locate the retainer against features inside the humidification chamber or avoid features (such as sensor(s), probe(s)) within the port. This means that the retainer can be safely removed and replaced without potential damage to the humidification chamber and while the tube winder port cap stays in position. The guide may be the aperture itself, including the shape and/or the edges of the aperture. In some cases the tube winder may have further walls, protrusions and/or recesses, such as a wall surrounding the aperture inside the tube winder, to better guide the retainer.

In some cases the aperture is closable, or closes automatically, if the elongate retainer is removed. For example a resilient living hinge may close the aperture if the elongate retainer is not present. This maintains the full closure of the port.

For purposes of summarizing the disclosure and the advantages achieved over the prior art, certain objects and advantages are described herein. It is to be understood that not necessarily all such objects or advantages need to be achieved in accordance with any particular example. Thus, for example, those skilled in the art will recognize that the disclosure may be embodied or carried out in a manner that achieves or optimizes one advantage or group of advantages as taught or suggested herein without necessarily achieving other objects or advantages as may be taught or suggested herein. All of these examples are intended to be within the scope of the disclosure herein. These and other examples will become readily apparent to those skilled in the art from the following detailed description having reference to the attached figures, the disclosure not being limited to any particular disclosed example(s).

### BRIEF DESCRIPTION OF THE DRAWINGS

These and other aspects of the present disclosure will be described with reference to the following drawings, which should be considered illustrative but not limiting.

FIG. 1 is a diagram of an example respiratory assistance system that includes a gases source such as a ventilator and a humidification device.

FIG. 2 is a perspective view of a humidification chamber mounted on a mounting portion of the humidification device with a tube winder mounted to the humidification chamber.

FIG. 3 is side view of the humidification chamber of FIG. 2.

FIG. 4 is a plan view of the humidification chamber of FIG. 2.

FIG. 5 is a perspective front view of a tube winder.

FIG. 6 is a perspective back view of the tube winder of FIG. 5.

FIG. 7 is a plan view of the tube winder of FIG. 5.

FIG. 8 is a back view of the tube winder of FIG. 5.

FIG. 9 is a side view of the tube winder of FIG. 5.

FIG. 10 is a back view of a tube winder.

FIG. 11 is a horizontal plane cross section of the tube winder of FIG. 10.

FIG. 12 is a vertical cross section of the tube winder of FIG. 11.

FIG. 13 is a front perspective view of a retainer.

FIG. 14 is a back perspective view of the retainer of FIG. 13.

FIG. 15 is a plan view of the retainer of FIG. 13.

FIG. 16 is a side view of the retainer of FIG. 13.

FIG. 17 is a front perspective view of a retainer.

FIG. 18 is a back perspective view of the retainer of FIG. 17.

FIG. 19 is a front view of the retainer of FIG. 17.

FIG. 20 is a front detail of the connection between a tube winder and a humidification chamber.

FIG. 21 is a side detail of the connection between a tube winder and a humidification chamber as shown in FIG. 20.

FIG. 22 is an exploded view of the retainer, tube winder, humidification chamber.

FIG. 23 is a perspective view of the inside of a port.

FIG. 24 is a plan view of the port of FIG. 23.

FIG. 25 is a cross-section view of the port of FIG. 23, a tube winder connected to the port, a humidification chamber and a retainer.

FIG. 26 is a perspective view of the inside of a port with an alternative protrusion.

FIG. 27 is a plan view of the port of FIG. 26.

FIG. 28 is a cross-section view of the port of FIG. 26, a tube winder connected to the port, a humidification chamber and a retainer.

FIG. 29 is a front perspective view of a tube winder mounted on the humidification chamber.

FIG. 30 is a back perspective view of a tube winder of FIG. 29.

FIG. 31 is a back perspective view of a tube winder with wound tube mounted to the humidification chamber with the retainer in place. Internal features of the humidification chamber are shown in broken line.

FIG. 32 is a side view of the tube winder and humidification chamber of FIG. 31. Internal features of the humidification chamber are shown in broken line.

FIG. 33 is a plan view of the tube winder and humidification chamber of FIG. 31. Internal features of the humidification chamber are shown in broken line.

FIG. 34 is a first perspective view of an example retainer.

FIG. 35 is side view of the example retainer of FIG. 34.

FIG. 36 is a second perspective view of the example retainer of FIG. 34.

FIG. 37 is a first perspective view of a further example retainer.

FIG. 38 is a second perspective views of the example retainer of FIG. 37.

FIG. 39 is a plan view of an alternative tube winder.

FIG. 40 is a cross-section of the tube winder of FIG. 39 along line B.

FIG. 41 is a cross-section of the tube winder of FIG. 39 along line A.

FIG. 42 is a perspective view of the tube winder of FIG. 39 from below.

FIG. 43 is a perspective view of a retainer with a tube retainer connected to a tube winder.

FIG. 44 is a side view of the retainer and tube winder of FIG. 43.

### DETAILED DESCRIPTION

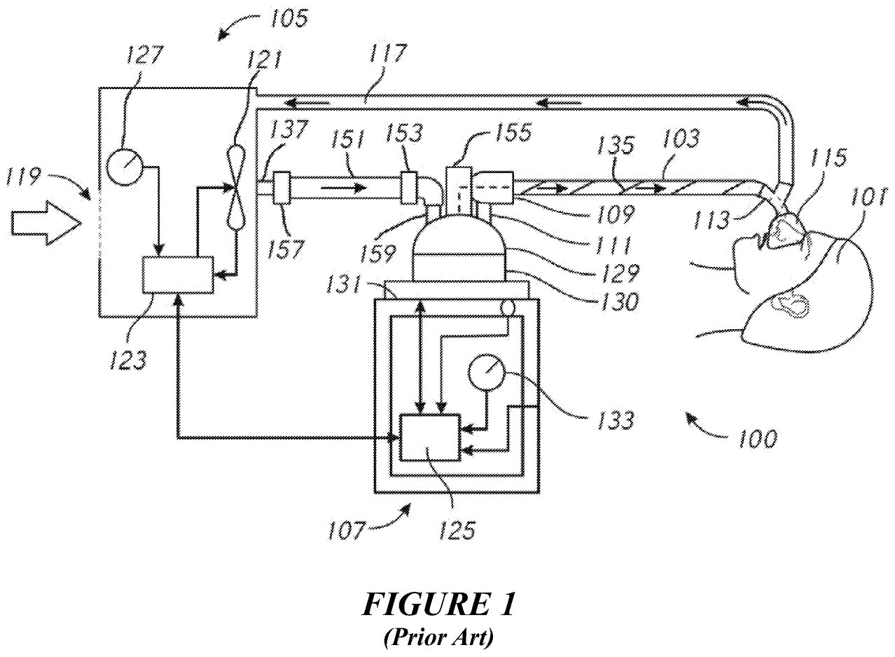

FIG. 1 is a diagram of an example respiratory assistance system 100 that may be used to provide respiratory gases to a patient 101. The respiratory assistance system 100 may comprise a gases source 105 in fluid communication with a patient interface 115 via an inspiratory conduit 103 and an expiratory conduit 117. The inspiratory conduit 103 and the expiratory conduit 117 may be connected to the patient interface 115 via a wye-piece connector 113. In some configurations, the gases source 105 comprises a ventilator. In some configurations, the gases source 105 comprises a blower 121 configured to draw air or other gases through an inlet 119 and deliver the air or other gases through a source outlet 137. In some cases, the gases source 105 is remote from the respiratory assistance system 100 and the gases source is provided by a conduit connected between the respiratory assistance system 100 and an outlet of a gases distribution system. An electronic controller 123 may control a speed of the blower 121 in response to input to the electronic controller 123, including but not limited to user input supplied via a user interface 127. The electronic controller 123 may also comprise, or be arranged to be controlled by, predetermined control algorithms that control the blower speed based on predetermined methodologies and/or based on data inputs from suitable sensors within the respiratory assistance system 100.

In the configuration shown, the respiratory assistance system 100 also comprises a humidification device 107 that is configured to condition respiratory gases provided to the patient 101. The humidification device 107 may be positioned in the fluid communication path between the ventilator 105 and the patient interface 115 to heat and/or humidify respiratory gases prior to delivery via the inspiratory conduit 103 to the patient interface 115. The humidification device 107 comprises a humidification chamber 129 that contains a liquid 130 and a heater 131 adjacent to the humidification chamber 129 to heat the liquid 130 to produce vapor that humidifies respiratory gases passing over the liquid 130. In some configurations, the inspiratory conduit 103 comprises a heater wire 135 configured to maintain a temperature of gases delivered through the inspiratory conduit 103 to the patient interface 115 and to reduce formation of condensate in the inspiratory conduit 103. An electronic controller 125 may control a duty cycle of the heater 131, a duty cycle of the heater wire 135, and/or at least a portion of the function of the electronic controller 123, in response to input to the electronic controller 125, including but not limited to user input supplied via a user interface 133. In some configurations, the electronic controller 123 may control at least a portion of the function of the electronic controller 125.

The humidification device 107 may be connected to the ventilator 105 by a supply conduit 151. In particular, a source connector 157 of the supply conduit 151 may be connected to the source outlet 137, and a humidification device connector 153 of the supply conduit 151 may be connected to an inlet port 159 of the humidification chamber 129. An inspiratory connector 109 of the inspiratory conduit 103 may be connected to an outlet port 111 of the humidification chamber 129, so that gases delivered to the humidification chamber 129 from the ventilator 105 through the supply conduit 151 can continue on through the inspiratory conduit 103 to the patient interface 115. In some configurations, the inspiratory connector 109 may be configured to be connected to a mounting portion 155 of the humidification device 107, so that electrical signals can pass from the humidification device to the inspiratory conduit 103 through an electrical connection provided by the inspiratory connector 109. As an example, but without limitation, the electronic controller 125 may control the duty cycle of the heater wire 135 via the electrical connection provided by the inspiratory connector 109. In some cases, the humidification device 107 is mounted on or combined with the gases source 105. The controllers 123, 125 and/or user interfaces 127, 133 may be shared between the devices.

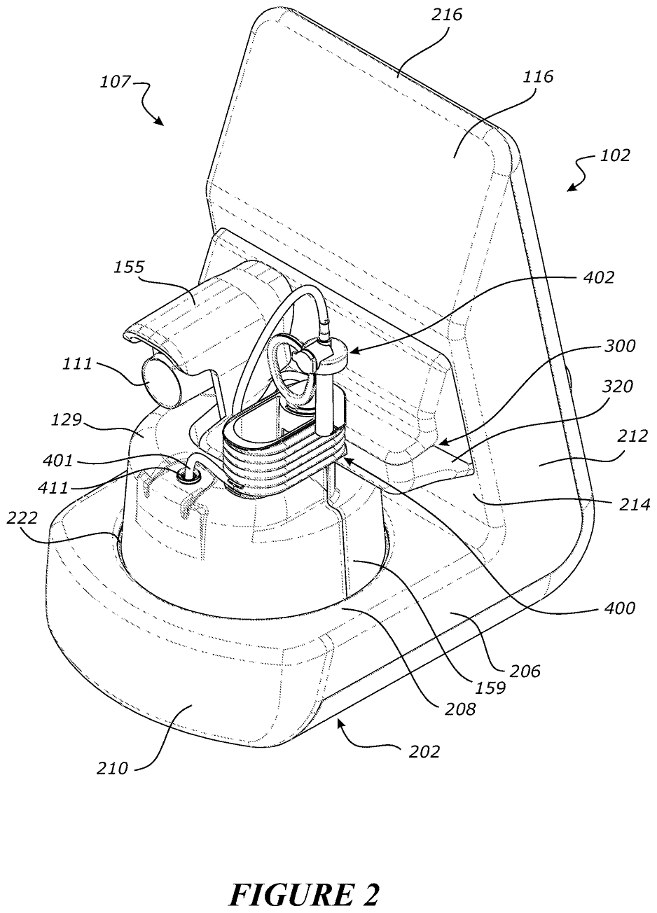

An example humidification chamber 129 is shown in FIGS. 2, 3 and 4. These show examples where the humidification device 107 comprises a humidification base 102 to which a humidification chamber 129 is connectable. The connection may be made by the humidification chamber being slid onto the humidification base 102. The humidification chamber 129 generally comprises an inlet 159 and an outlet 111 and is configured to be installed on a heater plate of a heater base 102 of a humidification device 107, such as humidification device 107 of FIG. 2. The heater base 102 may support the humidification chamber 129 and provide electrical and/or pneumatic connections to the humidification chamber 129 and/or comprise a controller for the humidification device 107. The humidification chamber 129 is configured to hold a volume of a liquid, such as water. The chamber 129 can include an opening 411, for the connection of a tube such as a liquid tube 401. The liquid tube 401 can extend from the chamber 129. In some configurations, the liquid tube can connect to a spike for a water bag. In some configurations, the liquid tube can be integrally formed with or permanently coupled to the chamber 129. The spike can be coupled to the liquid tube via an adhesive, sonic welding, an interference fit, or any other suitable means. In some examples, the spike includes a vent. If the spike is inserted into, for example, a plastic, collapsible bag, the vent is plugged. However, if the spike is inserted into a rigid container, such as a glass bottle, the vent is open and allows air to enter the container to help reduce or prevent negative pressures in the container. The vent can include a filter that is permeable to gases but impermeable to liquids. The humidification chamber 129 and the humidification base 102 may have one or more electrical, mechanical or pneumatic connections. These may allow electrical signals to be transmitted between the chamber and base, allow for alignment of the chamber and base and or allow for liquid or gas flows between the chamber 129 and base 102. In some cases the connections are be made at the outlet port of a humidification chamber. For example a connector may be configured to provide connections between the humidification chamber, the humidification base and the connector at the outlet port.

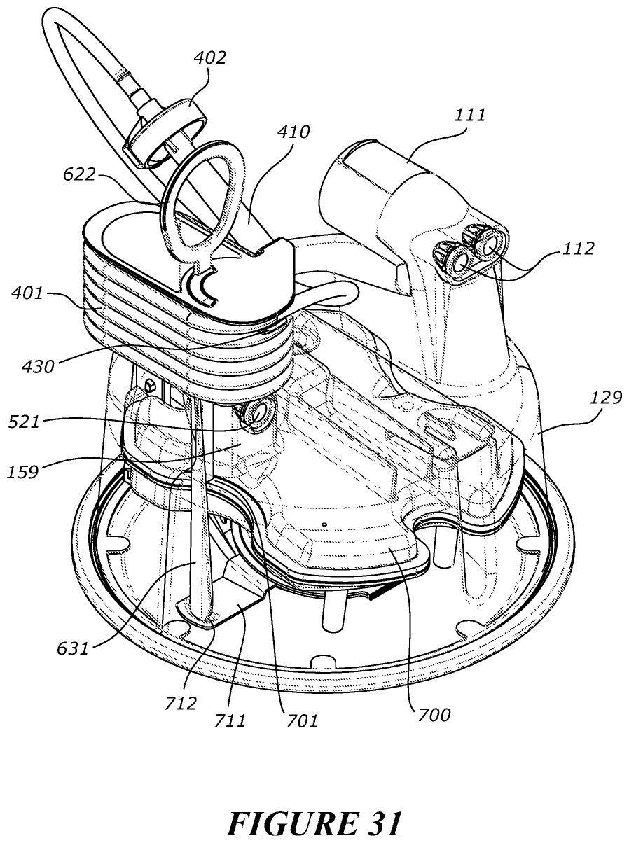

In use, the liquid tube conveys a liquid, for example, water, from a liquid source, such as a water bag, saline bag, or the like, to the chamber 129. The heater plate heats the chamber 129 and causes at least some of the chamber 129 contents to evaporate. In some examples, the humidification chamber 129 can include features to help reduce the likelihood of the level of liquid in the chamber 129 from exceeding a particular level. For example, the chamber 129 can include one or more floats 700, 701 (shown in FIG. 31).

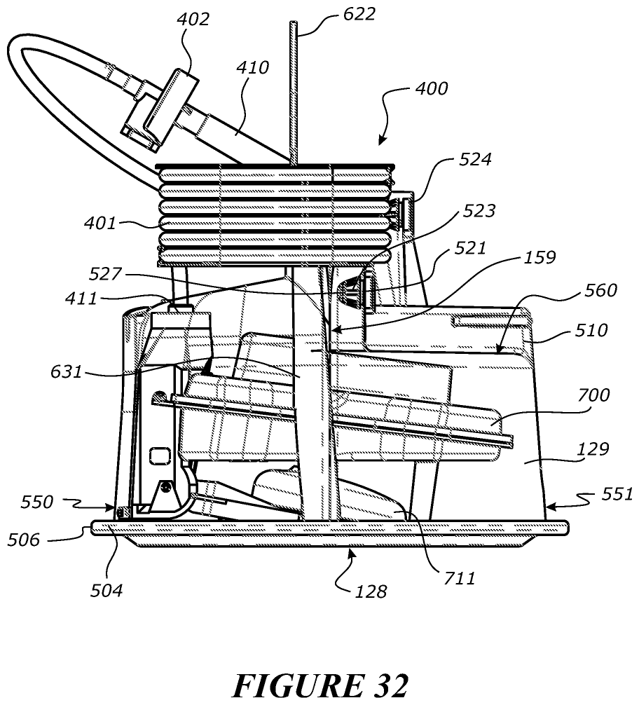

The floats rise and fall with the level of liquid in the chamber 129. When the liquid level reaches a certain level, the floats 700, 701 obstruct or block the port that is connected to the liquid tube 401 to stop or slow further ingress of liquid into the chamber 129. Other similar features also can be used. In a preferred example, a plurality of floats 700, 701 are used, each float adapted to stop the further ingress of liquid into the chamber 129. To this end, a second float provides a backup or safety mechanism, thereby further reducing the likelihood of the chamber 700, 701 overfilling. FIG. 32 illustrates an example of such a chamber 129 having a primary float 700 and a secondary float 701.

The chamber 129 is configured to connect to a breathing circuit assembly which can include a supply conduit, an inspiratory conduit, and, in some configurations, an expiratory conduit. A gases supply end of the supply conduit is configured to connect to an output of a gases supply and a chamber end of the supply conduit is configured to connect to the inlet 159 of the chamber 129. A chamber end of the inspiratory conduit is configured to connect to the outlet 111 of the chamber 129, and a user end of the inspiratory conduit is configured to connect to the user via an interface, for example.

In use, gases flow from the gases supply through the supply conduit and into the chamber 129 via the inlet 159. The gases are humidified within the chamber 129 and exit the chamber 129 through the outlet 111. The user inhales humidified gases supplied through the inspiratory conduit and may exhale into the expiratory conduit. The inspiratory conduit and/or expiratory conduit can include a heating element, for example, a heating wire, to help maintain the gases at a desired temperature and to reduce the likelihood of significant condensation formation in the conduits.

Before use, an operator, such as medical personnel, must correctly connect the various components to set up the system. Because of the variety of components and number of connections that must be made, set-up of the system can be a complex process that requires special training to complete properly. The humidification system can include various features as described herein to simplify the set-up process and reduce the likelihood of an incorrect set-up. In some cases, certain usability features advantageously can help reduce the total number of steps and time required during the set-up process. Some features described herein also can help make set-up more intuitive for the user, which can reduce the need for specialized in-service training.

An example of the heater base 102, as part of the humidification device 107, is illustrated in FIGS. 2, 3 and 4. In the example, the heater base 102 includes a base portion 202. The base portion 202 can include the heater plate which optionally sits beneath the humidification chamber 129, in use. The heater base 102 can include a spine 204. The spine 204 extends upwardly from a rear portion of the base portion 202. The base portion 202 includes side surfaces 206, a top surface 208, and a front surface 210. The spine 204 includes side surfaces 212, a front surface 214, and an upper surface 216. The upper surface 216 can include a display 116 and/or controls. For example, various dials, switches and other input means may be used to control operation of the device. Additionally or alternatively, the display 116 may be a touch screen display. The display 116 may display parameters of the system, warnings in the event of any errors or malfunctions or prompts where user action is required, etc. Where the display 116 is a touch screen display, the display 116 may be used to present information to a user and receive inputs from a user, at least in part.

The humidification chamber 129 can include a body formed of plastic with a base plate 504 sealed thereto that is heat conductive. In some cases, the base plate 504 of the humidification chamber 129 includes a lip 506 that protrudes beyond an outer perimeter of the body. In some cases the humidification chamber 129 is configured to be installed on the heater base 102 so that the base plate 504 of the humidification chamber 129 contacts the heater plate of the heater base 107. The humidification chamber 129 is adapted to hold a volume of liquid, such as water, that can be heated by heat conducted through the base plate 504 from the heater plate. Seals and/or grommets may be positioned on the humidification chamber 129. In some cases the seals/grommets are positioned on the rear of the humidification chamber 129 (i.e., the side facing towards the spine 204, in use) so as to seal connections between the humidification chamber 129 and the base 102 when connected.

In some cases the heater base 102 includes an inner chassis 222. The inner chassis 222 generally encircles the heater plate. The inner chassis 222 also includes a rim edge. The rim edge of the inner chassis 222 is positioned generally below the rim edge of the upper surface 208. A groove is formed between the rim edge of the upper surface 208 and the rim edge of the inner chassis 222. The groove can have a thickness of, for example, about 4 mm. The thickness of the groove is large enough to accommodate the lip 506 of the humidification chamber 129 and small enough to significantly limit a generally vertical range of motion of the lip 506 (and the associated humidification chamber 129) relative to the heater base 202. In some cases, the thickness of the groove is large enough to accommodate various configurations of chambers. In some cases, the diameter of the groove is large enough to accommodate various configurations of chambers.

In some cases the humidification chamber 129 includes gripping portions. The gripping portions can advantageously allow the user to grip the humidification chamber 129 more easily when installing or removing the humidification chamber 129 from the heater base 102.

The humidification chamber 129 can comprise a handle. The handle can be secured to the humidification chamber 129 in any suitable manner. The humidification chamber 129 may include a pair of bridges and a pair of fins, with one of the bridges and one of the fins on each side of the humidification chamber 4. Advantageously, by using snap together assembly, the handle can be secured to the humidification chamber 129 without the use of adhesives, solvents, or the like. Other configurations are possible, however.

The heater base 102 can include a coupling portion coupled to the front surface 214 of the spine 204. In some cases, the coupling portion is a cartridge 300 as shown in FIG. 2. The cartridge 300 can include or support various sensors, probes, sensor wire connectors, heater wire connectors, and/or other features. In some cases, the cartridge 300 includes features configured to mate with corresponding features on the humidification chamber 129.

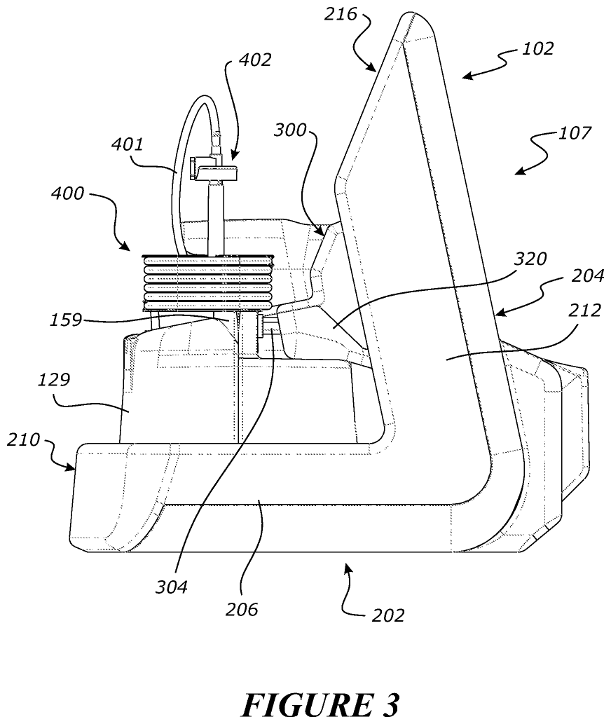

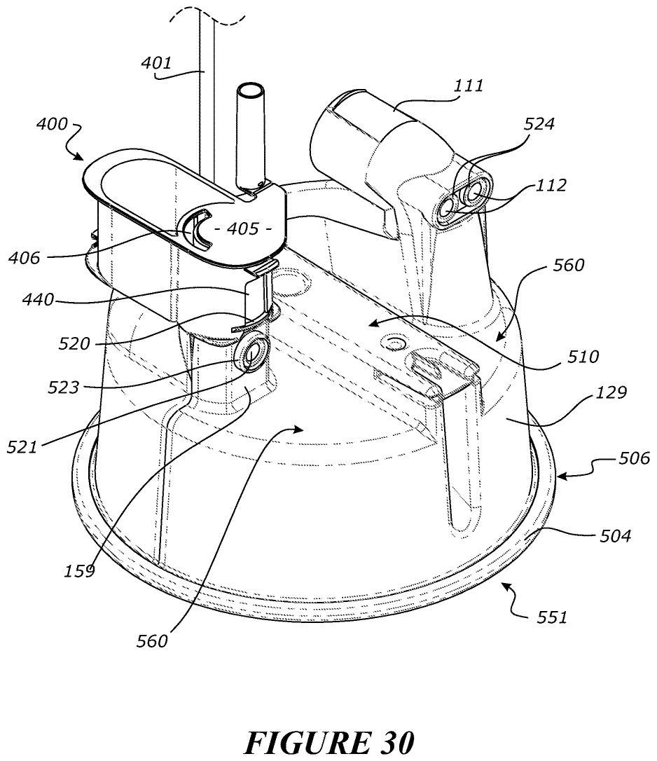

Any suitable components can be used as sensors. For example, thermocouples, resistance temperature detectors, fixed resistors and the like can be used as sensors. Sensors can be mounted directly onto the cartridge 300 or in probes or other housings that are mounted onto the cartridge 300. Such probes or other housings can also contain components other than sensors. The cartridge 300 can also be arranged in any suitable configuration or arrangement. FIG. 3 shows a cartridge 300 including a first probe 304 positioned on a first portion of the cartridge 300. In some cases, second and/or third probes are positioned on a second portion of the cartridge 300 to connect to openings 112. 521 on the humidifier (shown in FIG. 30). In some configurations, the first and second portions of the cartridge can be separated by a generally vertically extending plane. In some configurations, the generally vertically extending plane substantially bisects the heater base 102. In some configurations, the generally vertically extending plane substantially bisects the humidification chamber 129 when the humidification chamber 129 is positioned on the heater base 102 for use. The probes 304 extend forward from the cartridge 300. The probes 304 have thermistors mounted at the tips. Other configurations can be used, and other types of sensors can be used. In some cases probes have multiple sensors.

In some cases, the inlet port 159 and/or the outlet port 111 include one or more openings 112, 521 extending through the wall of the respective port. In the example shown in FIG. 30, the inlet port 159 of the humidification chamber 129 includes one opening 521 extending through a rear wall of the inlet port 159. The outlet port 111 includes two openings 112 extending through a rear wall of the outlet port 111. As shown, at least a portion of the each of the inlet port 159 and the outlet port 111 can have a respective flattened or contoured region. The openings 521, 112 may extend through the flattened or contoured regions. The flattened or contoured regions generally surround the openings 112, 521. Different numbers of openings and/or corresponding probes may be used.

The openings 112 in the outlet port 111 are configured to receive the probes when the humidification chamber 129 is installed on the heater base 102. Similarly, the opening 521 in the inlet port 159 is configured to receive probe 304 when the humidification chamber 129 is installed on the heater base 102. One or more of the probes may extend into the inlet port 159 and/or the outlet port 111. In some configurations, one or more of the probes extends into a portion of the humidification chamber 129 other than the inlet port 159 or the outlet port 111. In some configurations, one or more of the probes extends into a portion of the humidification chamber 129 other than the inlet port 159 or the outlet port 111 while one or more of the probes extends into one or more of the inlet port 159 and the outlet port 111. The probes extend into the outlet port 111 and inlet port 159 along axes parallel to an insertion axis along which the humidification chamber 129 is installed on the heater base 102. Mounting the probes on the cartridge 300 can advantageously allow for reuse of the sensors for multiple instances of the humidification chamber 129.

In some cases, the thermistor of the any one or more of the probes, such as probe 304 is configured to sense temperature of gases in the gas flow path. Multiple probes and/or sensors may be configured to sense a flow rate of gases in the gas flow path using a temperature flow measurement approach. In this approach, one of the thermistors functions as a reference sensor that measures the flow temperature at the sensing location and the other thermistor, which can be a heated thermistor, is heated to a preset temperature differential above the flow temperature. In some cases, the heated thermistor is heated to a set temperature, e.g., at or about 160 degrees Celsius. In some applications, a resistor can be used to heat the thermistor instead of using a heated thermistor. In some applications, a resistor can be used instead of a thermistor. In some configurations, all of the thermistors can be both heated and non-heated thermistors. Flow velocity can be determined using the measured flow temperature, the known heat transfer characteristics of the heated thermistor and the power consumed to maintain the temperature difference between the second and third thermistors. Other techniques also can be used. For example but without limitation, constant power can be provided to the thermistors and the heat conducted into a nearby thermistor can be used to determine the rate of flow. Other types of sensors are also possible.

As shown in FIG. 30, grommets 523, 524 can be inserted in the opening 521, 112 so as to cover at least the surface of the opening 521 and providing a protected passageway for the probe and/or sensor through the grommet. In some cases, alternative seals may be used instead of grommets. The grommets 523, 524 can at least substantially pneumatically seal the opening 112, 521. The grommets 523, 524 help isolate the gas flow path through the humidification chamber 129 from ambient while using the probes 304. Accordingly, in the illustrated configuration, the grommets 523, 524 define a barrier that reduces the likelihood of fluid or gas passing through the openings 112, 521. In some applications, at least one of the grommets 523, 524, and preferably all of the grommets 523, 524, also can be generally resistant to the passage of vapor. The grommets 523, 524 can be configured to receive the probes 304. In some configurations, the grommets 523, 524 allow the probes 304 to detect properties of gases flowing through the humidification system while remaining substantially pneumatically sealed from the gas flow path. The grommets 523, 524 advantageously allow the probes 304 to function without being in direct contact with gases in the gas flow path, so the probes 304 can be reused and do not require cleaning between uses.

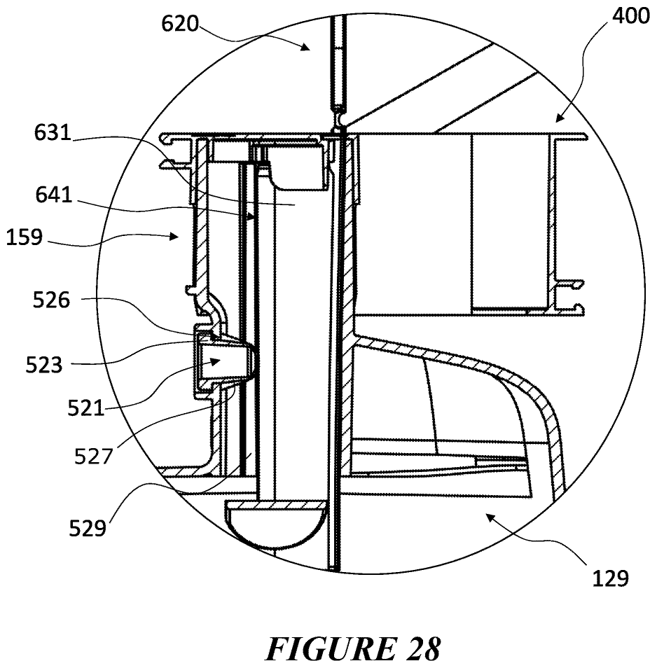

The grommets 523, 524 can be formed from any suitable material. In some applications, the grommets 523, 524 are formed from a resilient or flexible material. In some applications, one or more of the grommets 523, 524 can be formed of a material with a Shore-A hardness of between about 20 and about 80, and more preferably between about 40 and about 60. In some applications, one or more of the seals 523, 524 can be formed of Silicone, polyethylene, or thermoplastic polyurethane. The grommets 523, 524 may comprise a cap portion 527 (shown in FIGS. 28 and 32). The cap portion 527 may form a closure volume. In some cases the grommets 523, 524 are arranged so that the closed volume extend into the ports 111, 159. A probe 304 placed in the grommets 523, 524 may thus extend into the ports 111, 159 while the cap portion 527 protects the probe from contact with fluid passing through the port 111, 159.

In some cases, when one of the probes 304 is inserted into one of the grommets 523, 524, the one of the grommets 523, 524 stretches to accommodate the one of the probes 304. In some configurations, when one of the probes 304 is inserted into one of the grommets 523, 524, the one of the grommets 523, 524 conforms to the shape of the one of the probes 304. As the amount of stretch to accommodate the probes increases, the seal material becomes thinner. In some configurations, different regions of the grommets 523, 524 can be differing degrees of stretchability. In other words, some portions of the grommets 523, 524 are more elastic than other portions of the grommets 523, 524. This can advantageously improve the reactivity and accuracy of the sensor, increase the contact area between the sensor and the seal, and/or more securely hold the seal in the aperture.

The openings 112, 521 in the inlet port 159 and the probes 304 can be configured and can be positioned to improve the accuracy of the sensors. For example, positioning the probes 304 closer to the center of the gas flow path through the inlet port 159 can increase accuracy. Positioning the probes 304 closer to the center of the gas flow path can also help reduce errors that may be introduced if the probes 304 are too close to the wall of the inlet port 159. For example, positioning the probes 304 too close to the wall of the inlet port 159 can introduce wall effects, boundary layer effects, error due to thermal coupling, and/or other potential sources of error. However, it has been found that positioning the probes 304 too close to one another also can cause errors due to heat contamination.

Therefore, in some applications, it is desirable to maximize lateral displacement of the probes 304 from one another to reduce heat contamination while also maintaining sufficient proximity to the center of the inlet port 159 and sufficient distance from the wall of the inlet port 159 to improve accuracy and reduce wall effects and other potential sources of error. These requirements may increase the possibility of contact with other components in the port 159 because the probes 304 cover more space within the port 159.

In some cases, the lengths of the probes 304 are selected to allow the thermistors or other sensors to be positioned near the center of the gas flow path through the inlet port 159 and the outlet port 111. The axial lengths of the probes 304 and the sizing of the grommets 523, 524 can be interrelated to help promote more uniform stretching of the grommets 523, 524.

In some cases, one or more of the probes 304 are mounted on a flexible mount, such as a spring or resilient member. For example a first probe 304 may be mounted on a resilient member, and further probes 304 may be mounted on a resilient member. A single resilient member can support one probe in some configurations. In some configurations, a single resilient member can support two probes. In some configurations, a single resilient member can support two or more probes.

In some cases cartridge 300 comprises probes 304, where flanges of the probes 304 are generally D-shaped. The D-shape of flanges can allow the probes 304 to be positioned at the desired spacing from one another while providing enough support to the probes 304 so that they do not collapse or fold toward one another during engagement of the humidification chamber 104 with the cartridge 300.

The cartridge 300 and at least one portion of the humidification chamber 129 can have a coupling configuration. In some configurations, the cartridge 300 and an upper portion of the humidification chamber 129 can have a coupling configuration. The coupling configuration can promote correct and easy installation of the humidification chamber 129.

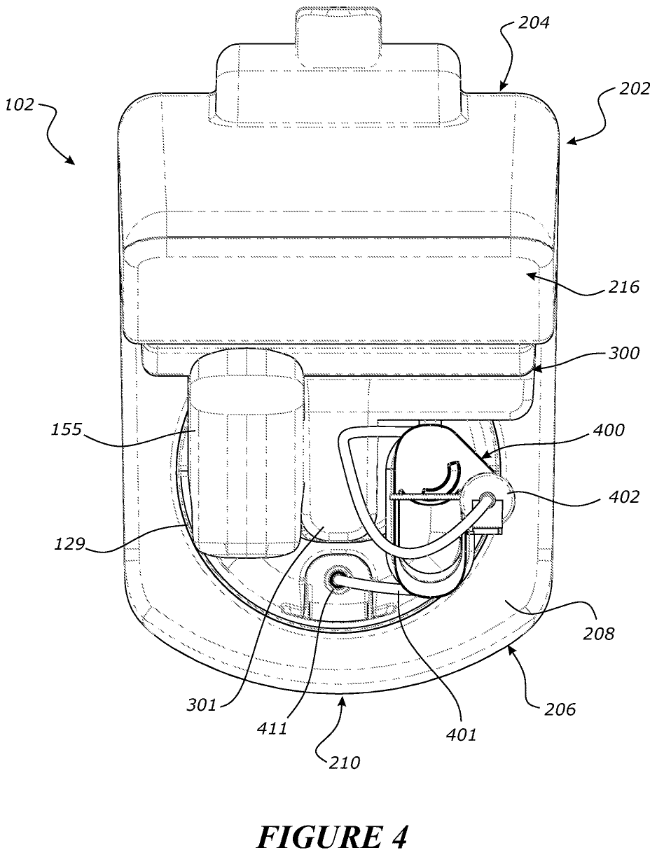

As shown in FIGS. 2, 3 and 4, the cartridge 300 includes outer sidewalls 320. In some cases, (not shown) the outer sidewalls 320 may extend beyond the tips of the probes 304. In some cases, the cartridge 300 also includes a protrusion 301 as shown in FIG. 4. The protrusion 301 may be configured to extend towards the centre of the humidification chamber 129 so as to occupy a central channel of the humidification device 107. As shown a surface may extend between the fins, linking them to form a single protrusion. The protrusion may be defined by sidewalls. The sidewalls may extend forward from the front face of the cartridge, each forming a side of the protrusion. The sidewalls can extend generally parallel to the cartridge outer sidewalls 320. A recessed portion can be formed between each sidewall 320 and the neighbouring protrusion sidewall. The probes 304 can be positioned between the protrusion sidewall and the cartridge sidewalls 320. In some configurations, at least one of the protrusion sidewalls and/or the outer sidewall 320 extends further outward from side surfaces 212 of the spine 204 than the distalmost ends of the probes 304. In some configurations, the protrusion 301 and the sidewall 320 adjacent to the probes 304 extend further outward from the side surfaces 212 of the spine 204 than the distalmost ends of the probes 304. In some cases, the protrusion 301 extends outwards from the cartridge. The protrusion 301 may be substantially central to the humidification chamber 129 and/or the base humidification base 102. The protrusion 301 may be configured to contact a top surface of the humidification chamber 129. The protrusion 301 may extend between the inlet 111 and outlet 159 ports. The protrusion 301 may extend further outwards than any other part of the cartridge 300. In some cases, the other parts of cartridge 300 may extend further than the protrusion. The protrusion 301 may extend further than the probes 304.

The cartridge may also comprise a mounting port 155. The mounting port 155 may be configured to pass electrical signals from the humidification device 102 (optionally through cartridge 300) to the inspiratory conduit. The inspiratory conduit may be connected to outlet port 111 and mounting port simultaneously. The mounting port 155 may extend from the cartridge as far, or further than the protrusion 301.

With reference to FIG. 30, the humidification chamber 129 may include a raised portion 510, the raised portion may be central to the humidification chamber. For example, extending along a radius of the top of the chamber towards the centre of the humidification chamber 129. In some cases, the raised portion 510 extends from the side of the humidification chamber 129 configured to connect to the cartridge and/or spine towards the centre of the humidification chamber 129. The humidification chamber 129 may include a groove located between the raised portion 510 and the outlet port 111 and/or a groove located between the raised portion 510 and the inlet port 159.

The protrusion 301, which may be a central protrusion, of the cartridge 300 may be configured to receive the raised portion 510 of the humidification chamber 129. The protrusion may be configured to slide into grooves on the humidification chamber 129. Alternatively, the protrusion 301 may extend over, or orientate around the raised portion 510 of the humidification chamber 129.

The shape and/or dimensions of the cartridge 300, such as the sidewalls 320 and the protrusion 301 may act as lead-in features to help guide the user in correct installation of the humidification chamber 129 on the heater base 102. Other guide features may alternatively be used. The shape of the cartridge 300 also help protect the sensors from damage that could be caused by improper contact with the humidification chamber 129. For example, if the user attempts to install the humidification chamber 129 with the front or a side of the humidification chamber 129 facing the cartridge 300 so that the openings 521 in the inlet port 159 and the outlet port 111 are not aligned with the probes 304, protrusion 301 will contact surfaces of the humidification chamber 129 to help reduce the likelihood of contact between the sensors and relatively hard surfaces of the humidification chamber 129.

The protrusion 301, or other cartridge features, and the humidification chamber 129 can also include features to help stabilize the humidification chamber 129 relative to the cartridge 300 and inhibit rotation, tilting, and/or yaw of the humidification chamber 129. For example, grooves can be configured to receive corresponding rails extending along the sides of the raised portion 510 of the humidification chamber 129. In some configurations, the grooves can be formed on the cartridge and the rails can be formed on the chamber. In either configuration, when the humidification chamber 129 is installed on the heater base 102 and coupled to the cartridge 300, the rails sit in the grooves. The coupling configuration of the rails in the grooves can help inhibit the humidification chamber 129 from excessive tilting.

In some cases, the cartridge 300 includes clips configured to engage and secure the humidification chamber 129. The body of the humidification chamber 129 can include corresponding recesses. The recesses can be configured to receive the clips when the humidification chamber 129 is installed on the heater base 102. The engagement of the clips with the recesses can provide a positive engagement feel to the operator to indicate to the operator that the humidification chamber 129 is fully installed on the heater base 102. The clips and recesses can also help promote proper installation of the humidification chamber 129 so that the probes 304 are properly inserted in the inlet port 159 and the outlet port 111.

To begin set-up, the operator installs the humidification chamber 129 on the heater base by sliding the chamber 129 onto the heater base. Engagement features such as a rim edge on the heater base can help hold the chamber 129 in place. The heater plate can be spring loaded in some configurations such that the base of the chamber 129 presses downward upon the heater plate and a protruding portion (not shown) of the chamber 129 can be captured between the heater plate and the rim edge. Features may be provided to prevent unwanted or accidental removal of the chamber 129 from the heater base. For example, a guard along a front portion of the rim edge may be provided that is depressed to enable the lower portion of the chamber 129 to engage access the heater plate and then the guard reverts to a non-depressed position once the chamber 129 is installed. This advantageously provides positive feedback that the chamber 129 is fully installed on the base.

In some configurations, the forwardmost portions of the rim edge (e.g., the portions of the rim edge that define an opening for insertion of the chamber 129) are configured with a raised or enlarged opening that ramps downward. The opening preferably comprises a lower surface that is elevated above an upper surface of the non-depressed guard. In such a manner, the opening provides a visual clue to the operator that the protruding portion 129 can be inserted into the opening. Further insertion of the chamber 129 into the opening causes the guard to be depressed and facilitates full insertion of the chamber into the heater base and can help guide the chamber 129 into place. Thus, these visual details can indicate to the operator that the chamber 129 slides into place under the rim edge. This can also help inform the operator that the guard can be depressed to later remove the chamber 129 from the heater base. Preferably, the chamber 129 has details to depress the guard when the operator attempts to remove the chamber 129 from the heater base. Moreover, by providing an uneven upper surface to the rim edge, the operator is less likely to believe that the chamber 129 should be placed atop the rim edge, resulting in poor thermal conductivity, because such a placement will lead to an uneven positioning of the chamber 129.

In some cases, a guard extends along a front portion of the base portion 202 of the heater base 102 and the rim edge. The guard can be depressed to enable the base plate 504 and the lip 506 of the humidification chamber 129 to contact the heater plate and the groove 226. The guard can be allowed to revert to a non-depressed position once the humidification chamber 129 has been installed. In the non-depressed position, the guard retains the humidification chamber 129 against inadvertent removal from or significant movement (e.g., significant forward movement) relative to the heater base 102. As shown in FIG. 2, the guard has a vertical dimension that shields at least the heater plate. In other words, when in the non-depressed position, the uppermost extent of the guard is vertically higher than the top of the heater plate. As such, the guard provides some degree of protection by serving as a shield to limit the likelihood of inadvertent human contact with the heater plate or other heated components of the system when the guard is in the nondepressed position. In some cases it may be advantageous to cover the inlet 159 and/or the outlet 111 when they are not in use. For example, before installation of the humidifier chamber 129 into the humidifier base 102, or for shipping and storage.

As shown in FIGS. 2, 3 and 4, and in other humidification devices the connection between the humidification base 102 and the humidification chamber 127 may involve orientation of the chamber and one or more connections. In some cases, cartridge 300 is not present. Instead, sensors and or protrusions may extend directly from the humidification base 102. This may lead to further unprotected, or adjustable probes or connections being present. The presence of these features can lead to the mounting of the humidification chamber 129 to the heater base 102 being able to move, dislodge or damage the heater base. This damage may require repair or replacement of the heater base and/or humidification chamber. In some cases, a user wants to protect the humidification chamber from any accidental contamination or spill, for instance through the inlet or outlet ports. In some cases, portions of the heater base 102 can protrude forward from the spine 204 or another vertical portion of the heater base 102 to make contact with the humidification chamber. These portions may assist with alignment of the humidification chamber or provide additional sensing locations. Therefore, it is advantageous for all the ports to remain capped, or substantially closed during installation and/or set-up of the humidification chamber. Therefore, it is also advantageous to reduce unnecessary contact between the humidification base 102 and the chamber 129.

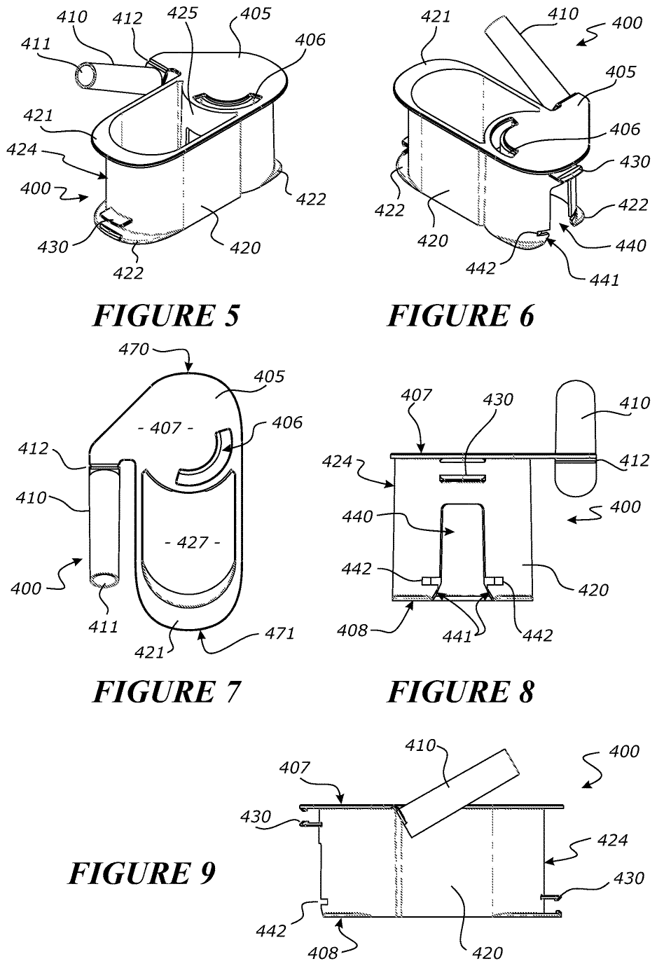

FIG. 5 shows a tube winder 400. The tube winder 400 has an engagement surface to engage the inlet (or outlet) port 159. The engagement surface is shown as completely surrounding the port; however, it may substantially surround the port, or partially surround the port in other cases. It has a side wall 420 with an outer surface 424 configured to support a wound tube. The tube winder 400 may act as a cover for the port when engaged.

In some cases, the tube winder 400 has a cover 405. The cover 405 may comprise a portion of upper surface 407. Alternatively, the cover may be arranged below and/or above the upper surface 407 (shown in FIG. 8). The cover 405 is configured to at least partially occlude, or fully occlude the port 159 when the tube winder 400 is mounted on the port 159. The cover 405 may have an aperture 406 to allow a retainer to pass through the tube winder 400 and into the port 159. The aperture 406 is arranged and/or configured to control an orientation of the retainer 620 relative to the port 111, 159. For example, as shown in FIG. 18, the aperture 406 may be dimensioned and shaped substantially the same as at least an outer profile or surface of the portions of the retainer 620 received therein. In some cases, the dimensions and/or shape may be slightly larger to accommodate said portions without excessive force being required. Consequently, movement of the retainer 620 is preferably restricted once fully inserted into the tube winder 400 such that it can only move in a direction aligned with the passageway, and when attached to the chamber, the port 111, 159. In some cases the cover 405 may be referred to as a lid, although the lid or cover 405 will typically only cover a portion of the port 159 to allow the retainer 620 to pass therethrough into the port.

In some cases, a flange may extend away from the upper 407 and/or lower 408 surfaces. In some cases, the flange may extend away from the side wall 420. The flanges are configured to support and/or retain the wound tube on the tube winder 400. The flanges may prevent or reduce vertical movement of the wound tube (upwards and/or downwards movement of the wound tube relative to the side wall).

The tube winder 400 assembly can include other or additional orientation and/or alignment features. For example, the tube winder 400 can include one or more projections or recesses that engage one or more corresponding recesses or projections associated with the port 111, 159 (preferably provided on an external wall of the port). Although the discussion may refer to the inlet port 159 or outlet port 111 it will be understood that the tube winder 400 may be configured to attach to either the inlet port 159 or outlet port 111 or both ports in some cases. In some cases the projections or recesses are configured so as to control a rotational orientation of the tube winder 400 relative to the inlet port 159 about an axis through the port 159. For example, FIG. 20 shows the tube winder 400 having a slot 440 in the sidewall. In this case the sidewall also forms the engagement surface. The slot 440 has corresponding recesses 442, configured to engage a rib 520 on the inlet port 159. The recesses 442 form a catch to secure the tube winder 400 on the inlet port 159. The catch may also prevent rotation of the tube winder 400. The rib 520 on the port 159 can further act as a stop to limit the extent to which the tube winder 400 can be inserted onto the inlet port 159. In FIG. 20 this is achieved because the rib 520 is secured in the recesses 442. Additionally, or alternatively, a projection such as on the tube winder 400 can engage recess in a wall, in some cases an external wall, of the port 159. The side wall 420 on either side of slot 440 may be resiliently deformable, having enough flexibility to expand over the rib to allow it to move into recesses 442 with downwards pressure. Sloped surfaces 441 at the mouth of slot 440 may urge or guide the slot 440 over and/or around the rib 520 to help this process.

In some cases, the tube winder 400 comprises an aperture lid (not shown) for aperture 406. The aperture lid can be connected to the body of the tube winder 400 via a hinge. This hinge may be in the form of a flexible arm of, for example, polymer-based material, that may be integrally formed with or fixedly joined to the tube winder 400. The aperture lid may be configured to engage the cover 405 with a snap-or friction-or other type of fit. The aperture lid may provide full closure of the aperture 406 when the retainer 620 is not present.

In some cases, the tube winder 400 can comprise a holder 410, such as a sleeve, for receiving and removably retaining a spike which may be provided on the liquid tube. The liquid tube may be a tube filled with a water or another humidifying agent feedset. A “feedset” or “feed set” may comprise components for supply of a humidifying agent to the humidification chamber. For example, the spike may be coupled or couplable to a liquid tube 401 that is coupled or couplable to the humidification chamber 129. The holder 410 can be connected to a main body of the port cap 400, preferably at or adjacent the cover 405.

The tube winder 400 can include a joining wall or side wall 420 extending between the upper and lower surface 407, 408. The side wall 420 preferably extends from at or near the perimeters or edges of the upper and lower surfaces 407, 408. The side wall 420 can extend beyond the upper and lower surfaces to provide greater side wall area. At least one opening can be provided in the side wall 420 such that the side wall 420 does not extend completely about the perimeters or edges of the tube winder. The opening(s) or window(s) may reduce the weight or improve usability of the tube winder 400.

The side wall 420 can define a space for accommodating at least a portion of flexible tubing such as liquid tube 401. More particularly, at least a portion of liquid tube coils may be wound about the outer surface 424 of side wall 420. The end of the liquid tube may then be placed in the holder 410 through opening 411. Winding the liquid tube 401 on the outer surface 424 of the sidewall 420 means that the tube is wound about the external surface of the tube winder 400, which is easier for manufacturing and for user access.

To assemble the tube winder 400 assembly to the chamber 129, the tube winder 400 may first be slid into engagement with the port 111, 159. The aperture 406 then provides a locating region or space for the retainer 620 to be slid into engagement with the tube winder 400 with the leg 631 of the retainer passing into the port and/or chamber. In some cases, the orientation features, described above on the tube winder 400, ensure a selected or particular orientation(s) of the tube winder 400. The aperture 406 then aligns or controls the orientation of the retainer 620 relative to both the tube winder 400 and the port 111, 159. In some cases, the retainer 620 may be inserted in the tube winder 400 before the tube winder 400 is engaged to the port 111, 159. In some cases, the retainer 620 may also have alignment or orientation features which engage or interact with corresponding features inside the port 111, 159. The shape and orientation of the tube winder 400 may also allow the humidification chamber 129 to be connected to the humidification base 102 while the tube winder 400 is connected. The surface of leg 631 may also be used to align the retainer relative to the aperture, for instance against wall 661, or the port itself. In some cases, upper surface 630 may also be used to align the retainer 620 relative to the aperture.

An operator has two options to remove the tube winder 400. In a first option the tube winder 400 assembly can be lifted from the chamber 129 by grasping the sides or ends of the tube winder 400. In some cases, the tube winder 400 can be removed rotationally by lifting the distal end of the elongate tube winder 400 to leverage the engagement surface away from the port 111, 159. Alternatively, the retainer 620 may be removed by the operator pulling on the pull tab 622 to remove only the retainer 620. In some cases, the tube winder 400 is held in place by the engagement surface. This allows the cover 405 of the tube winder 400 to remain over the port opening, even when the retainer has been removed to release the floats or components within the humidification chamber 129. This reduces the need to reinsert the retainer 620 which can help prevent damage caused by the retainer 620 to components inside the chamber 129 e.g., grommets 523, 524 covering sensor ports or openings 112, 521 into the chamber 129. This also means that setup can be completed while still having at least a partial cover to prevent unwanted spillage into the chamber 129. The user does not need to use a separate cover to ensure that port of humidifier is occluded. This may prevent objects or liquids from entering the port 111, 159 when the user is setting it up. This may be particularly relevant where the port 111, 159 is vertical.

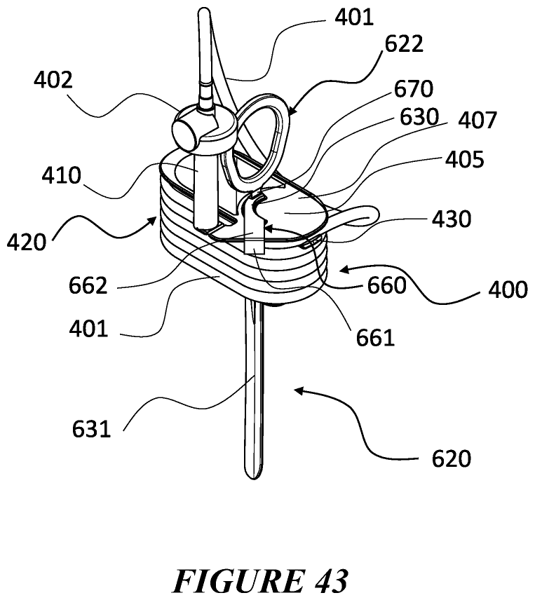

In some cases, the retainer 620 has an additional retaining device to prevent unwinding of the tube 401 until the retainer 620 is removed. The retainer 620 may have an additional retainer, for example tube retainer 660, shown in FIG. 43. The additional retainer may extend over the tube winder 400 and at least partially down the side wall 420. Because the liquid tube 401 is coiled at least partially around the side wall 420, the additional retainer ensures that a user is substantially prevented from connecting the liquid tube 401 to a water bag or other reservoir until the retainer 620 is removed. This is advantageous as the floats are “deactivated” with the retainer in position and so overfilling of the chamber can be prevented. The additional retainer may be a separate component. The additional retainer may be referred to as a tube retainer 660. The additional retainer may extend over a portion of the wound tube 401, for example to protect the tube 401 in transport. The portion may be substantially all the wound tube 401 or a majority of the wound tube 401. The additional retainer may be removable from the tube winder 400. For example, the additional retainer may be a clip which fits over the wound tube 401, or a band which fits over or around the wound tube 401.

FIG. 43 shows an example tube retainer 660 as part of the retainer 620. As shown, the tube retainer 660 extends away from the body of retainer 620 between the pull tab 622 and the leg 631. However, alternative positions may be used. The tube retainer 660 is shown when the retainer 620 is engaged with the tube winder 400. The tube retainer 660 extends across the upper surface 407 of the tube winder 400 and may extend across the cover 405 portion of the upper surface 407. A distal end of the tube retainer 660 is configured to retain the wound tube 401 on the side wall 420 of the tube winder 400. As shown in FIG. 43 the tube retainer 660 may be implemented by an extension portion 662, for example extending across the upper surface 407, and a retention portion 661 extending away from the upper surface 407 to prevent the wound tube 401 from being unwound from the sidewall 420. The retention portion 661 is shown substantially perpendicular to the upper surface 407 (or within 10 or 15 degrees of perpendicular) and extension portion 662 is shown substantially parallel to the upper surface 407, although this angle is not required. In some cases the retention portion 661 and extension portion 662 may be substantially perpendicular to one another.

The extension portion 662 shown in FIG. 43 extends in a direction towards the holder 410, or to a portion of the upper surface 407 having a larger radius. Alternatively the extension portion 662 may extend towards the clip 430 or any other portion of the edge of the upper surface 407. The extension portion 662 enables the placement of the retention portion 661. The location of the tube retainer 660 and therefore the retention portion 661 may be controlled by the position of the retainer 620. When the retainer 620 is secured to the tube winder 400 the position of the retention portion 661 may also be fixed with respect to the tube winder 400, so as to locate the retention portion 661 where required to retain wound tube 401. In some cases the wound tube 401 is now retained in multiple locations, by one or more of: clips 430 to secure it to the tube winder 400; tube retainer 660 to secure it when the retainer 620 is in place; and holder 410 to secure the end of the tube with water spike 402. The retainer 620 may be unitary, with the leg 631, pull tab 622 and tube retainer 660 all being connected. One or more of the connections between the parts of the retainer 620 may be made flexible, such as neck 670 shown between the pull tab 622 and the leg 631 and/or tube retainer 660. In some cases the tube retainer 660 may be separate, or separatable from the retainer 620 to allow it to be removed separately form the retainer 620, if desired.

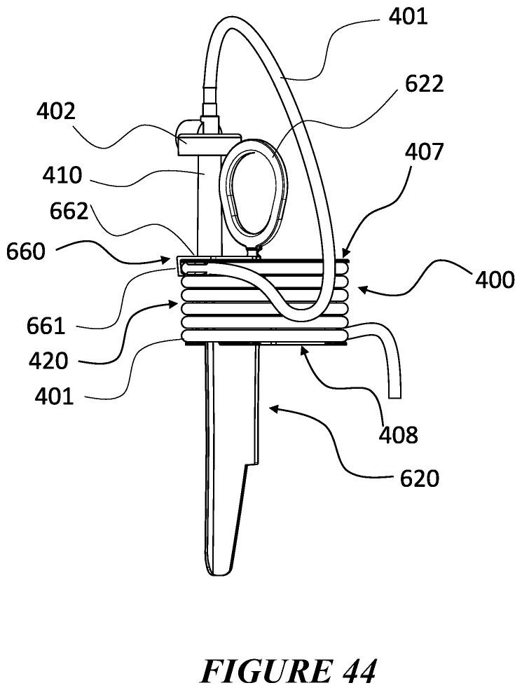

FIG. 44 shows a side view of the assembly of FIG. 43. Again, the retainer 620 is shown with the tube retainer 660 having an extension portion 662 and a retention portion 661. The extension portion 662 is shown substantially parallel to the upper surface 407 of the tube winder 400, with the retention portion 661 extending towards the lower surface 408 of the tube winder 400. As shown the retention portion 661 may be angled towards the tube 401 (or the underlying sidewall 420). The angle may be configured to be less than 10 or less than 5 degrees from vertical. This may improve the retaining of the tube 401. In other cases the retention portion 661 may extend perpendicular or outwards. The example of FIG. 44 shows the retention portion 661 extending substantially down substantially the distance of one winding of tube 401. This secures the tube 401, optionally without preventing a user removing the tube by applying sufficient force. In some cases the retention portion 661 extends further, for example halfway down the tube winder 400 or to bottom surface 408. This may provide improved securing of the tube 401 to the winder 400. The retention portion 661 may be releasably secured to the tube winder, for example at bottom surface 408 to better secure retainer 620. For example the retention portion 661 may clip to the bottom surface 408. The securing may be flexible, so as not to prevent removal, or to damage the tube winder 400 or tube 401 when a user removes the retainer 620 by applying a force to pull tab 622.

The tube retainer 620 may be varied in shape and/or size depending on the desired retainment. FIGS. 43 and 44 shown a substantially rectangular extension portion 661 extending from an end of the upper surface 630 of the retainer 620. The retention portion 661 is shown as substantially rectangular and is located at the distal end of the extension portion 661. In another example the extension portion 662 may be narrower. For example the extension portion 662 may maintain the width of the upper surface 630 of the retainer 620. In some examples the extension portion 662 may extend parallel to the direction of the or each end of the upper surface 630, forming a direct extension of the upper surface. In another example the extension portion 662 may extend over all or substantially all of the cover 405. In one example extending from each end of the upper surface 630 of the retainer 620 and covering the space in between. In a further example the tube retainer 660 may also or instead extend across all or part of body space 427. This may enable the tube retainer to secure the tube along a more distant part of the outer wall 420.

In some cases the retention portion 661 extends along the entire distal end of the extension portion 662. Alternatively there may be one or multiple retention portions 661 spaced along the edge of the extension portion 662. In another example there may be multiple tube retainers 660, each extending from the retainer 620 to retain the tube 401 in spaced locations about the tube winder 400. In another example the retention portion 661 may be shaped to provide retainment of tube 401. For example the retention portion 661 may be tapered to be wider and/or narrow at the distal end. For example the retention portion 661 may comprise a plurality of fingers.

In some cases, the edges 640, 641 or the circumference of the retainer 620 may be shaped to reduce the likelihood that it will shear against and dislodge or damage a grommet 523, 524 for a sensor and/or a sensor. The shape of the retainer 620 may also be configured to provide a close fit to the aperture 406 for orientation during insertion, or to guide the retainer 620 into position. Additionally, or alternatively, the width or circumferential extent of the retainer 620 may be reduced, also reducing the likelihood of damage to components of the humidification chamber. In some cases, portions of the retainer 620 may be shaped so as to weaken the retainer 620 and reduce its width, thereby reducing the likelihood that it will shear against and dislodge or damage a grommet 523, 524 for a sensor and/or a sensor.

FIG. 32 shows the tube winder assembly assembled with a chamber. As shown, the humidifying liquid (e.g., water) feed line (for example liquid tube 401) is shown coiled around the tube winder 400 with a holder 410 such as a sleeve retaining a spike of the liquid tube 401 in opening 411. The holder 410 prevents the spike from being able to puncture the liquid reservoir, or otherwise couple thereto until it is used by the operator. In some cases, the holder 410 merely supports or locates the end of the liquid tube 401, or the spike. The holder 410 may be attached to the upper surface 407 or lower surface 408 of the tube winder 400. The holder may be attached to the cover 405. The holder 410 may be attached to the retainer 400 with an angled surface so that the tube does not get snagged or caught when winding. The angle of the holder may be adjustable. In some cases, holder extends in the same direction as the winding direction.

The aperture 406 can act as a visual indicator as to where to insert the retainer 620. In some cases there is no other aperture or gap in the cover 405 into which the retainer 620 may be placed. This may be achieved by there being a single aperture 406. For example, the aperture 406 may have a width less than a thickness or profile thickness or cross-sectional shape of the retainer 620 so the retainer 620 can only insert in a single orientation. In other cases, a plurality of openings configured to safely support the retainer 620 may be present on cover 405, providing a selection of locations for insertion of the retainer 620. Consequently, damage to a grommet 523, 524 and/or a sensor may be avoided by only allowing insertion of the retainer 620 in a predetermined orientation that is offset from the grommet and/or sensor. In some cases, the cover 405 may have a closure such as a lid (not shown) to close the aperture when the retainer 620 has been removed. The closure may be of a size, commensurate with the size of the aperture 406. The closure may be flexibly joined to the tube winder 400 and biased into a closed position.

FIG. 5 shows an example of a tube winder 400. The tube winder 400 has a body comprising side wall 420 with an outer surface 424 configured to support a tube 401 (shown in FIG. 31). The tube body may be made of plastic. The tube 401 may be wrapped several times (i.e., several separate layers around the height of the tube winder and/or several layers deep) around the side wall 410 to allow a significant length of tube 401 to be stored. The tube winder 400 is configured to connect to a port 111, 159 on the humidification chamber 129. In some cases, the port is an inlet port 159. The port 111, 159 may be configured to have an opening along a substantially horizontal or vertical axis, or at another angle, for example an angle between 0 and 90 degrees relative to the plane of the humidification chamber base. In some cases, the tube winder 400 is connectable or configured to connect to a single port 111, 159. This means it does not connect to or engage with to a second port 111, 159. In some cases, it may not extend to or contact a second port 111, 159. This allows the remaining ports of the humidification chamber 129 to be unencumbered by the tube winder 400. It may also mean the tube winder 400 can be positioned more usefully, for instance without obscuring a raised portion 510 of the humidification chamber 129 and thereby providing space for other components. The raised portion 510 may follow a diameter of humidification chamber 129. The raised portion 510 may be in the centre of the humidification chamber 129. In some cases, the raised portion 510 may instead be a recessed portion. In some cases where the winder 400 is configured to connect to a single port 111, 159 the winder 400 is able to avoid connections around the port 111, 159 and/or the humidifier 129, particularly connections between the humidification base and the humidification chamber and/or between connections between other tubes and other inlets of the chamber.

FIG. 5 shows a portion of side wall 420 configured to extend around a port 111, 159 to form an engagement surface. In the illustrated case the engagement surface of the tube winder 400 with the port is also formed, in part, by an inner side wall 425 and the inner surface of side wall 420. However, in some cases the port connection may use other surfaces and/or inner walls, or a different type of connector, such as a clip or band may be used instead. As shown, the side wall 420 substantially surrounds the port, so that the supported tube 401 is supported to form one or more, or a plurality of, loops around the port. Although the side wall 420 is shown completely encircling the port in some cases it may have discontinuities and/or an open end, so long as it is configured to support and/or retain the tube 401. In some cases, it may have an open portion so as to only partially surround the port. The side wall 420 may define a shape of the tube winder 400 and/or the wound tube 401.

As shown in FIG. 7 the tube winder may be elongate. That is to say, a first dimension of the tube winder 400 (shown extending away from the cover 405, or port engagement end 470), is greater than a second dimension of the tube winder 400 (shown extending across the port). The third dimension of the tube winder 400 is a depth along port the tube winder 400 is configured to connect to. The elongate shape of the tube winder 400 allows the winder 400 to extend in substantially a single direction. In some cases, this means it can be orientated on the humidification chamber 129 so as to be directed away from components configured to engage the humidification base 102. In some cases, the orientation is towards, at least in part, a front of the humidification chamber 129, or parallel to a central axis of the humidification chamber 129. The elongate shape may be formed by the side wall 420. The elongate shape and/or the side wall 420 may be substantially rectangular. The elongate shape, when viewed from above, may be a stadium/discorectangle. The elongate shape and/or the side wall may have curved or chamfered corners. The stadium/discorectangle shape and/or the curved or chamfered corners may help to avoid kinks when winding the tube 401 or make the liquid tube winding easier. The elongate shape and/or the side wall may have at least one end with a flat surface. As between ends 470, 471 the side wall has portions that are flat surfaces, shown on opposing sides of the tube winder 400. The flat surfaces may provide gripping locations for the user to hold the tube winder 400.

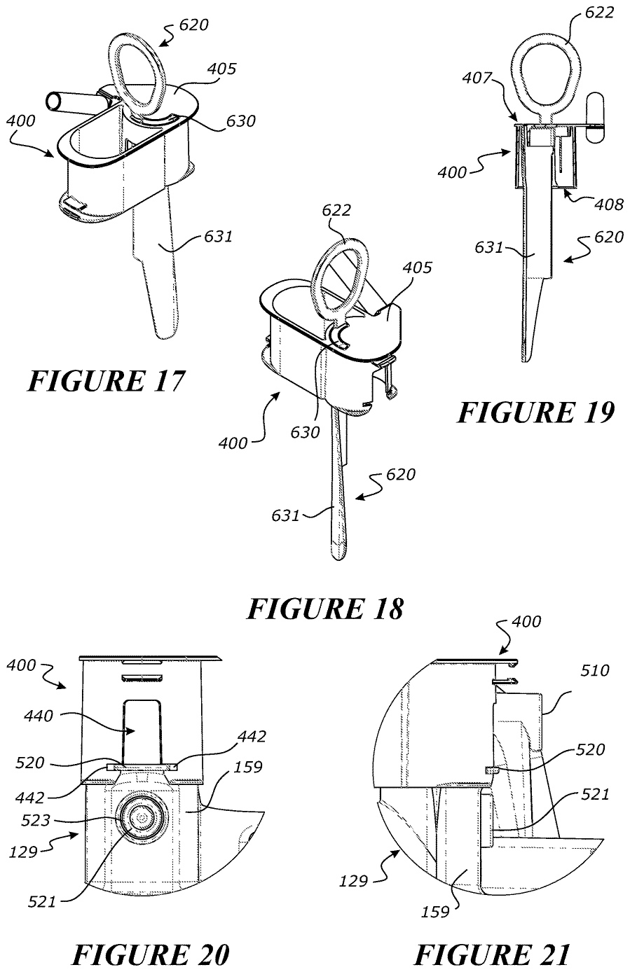

FIG. 6 shows the other end of the tube winder 400 (that is to say the opposite end along the elongate axis from FIG. 5). This end is configured to have an engagement with the port 111, 159, such as the engagement surface on the inside of the side wall 420 and inner side wall 425. In some cases, cover 405 is present to substantially occlude or reduce access to the port opening. In some cases, cover 405 extends substantially continuously across at least the opening of the port when the tube winder 400 is connected. An aperture 406 may be present to allow a retainer 620 (shown in FIG. 17) to pass through the tube winder 400 and into the port 111, 159 and/or humidification chamber 129.

A holder 410, shown as a sleeve may extend from, or connect to, the tube winder 400. The holder 410 may form part of the tube winder body, be permanently connected to the body or be detachable (or reversibly detachable) from the body. It may be arranged or positioned to avoid interference with any components on the humidification chamber and/or base. The holder 410 may be configured to support a spike at the end of the liquid tube 401 to be wound about the tube winder 400. The spike may be supported when it is placed in opening 411 of the holder 410. Although shown extending from a side of the cover 405, the holder 410 may be otherwise arranged on the tube winder 400. In some cases, a hinge 412 may be used to allow positioning of the holder 410. Hinge 412 may be formed by a reduction in thickness of the tube winder body and/or may be a living hinge.

FIG. 7 shows a plan view of the tube winder 400. Again, a distal end 471 of the tube winder 400 can extend from the port engagement end 470 so as to form the elongate winder 400. The cover 405 may extend over substantially all of the of the tube winder 400. Alternatively, as shown, the cover 405 may extend only partially over the tube winder. The cover 405 may extend over substantially all of, or a majority of, the port opening. In some cases, a body space 427 may be present between the distal end of the side wall 420 and the port engagement surface. This body space 427 may provide a suitable grip or holder for the tube winder 400 allowing a user to easily pull or move the tube winder 400.

FIG. 7 also shows a view of the aperture 406 for a retainer 620. The aperture 406 may be sized dependent on the size and/or shape of the retainer. In the illustrated case the aperture 406 is arcuate or curves so as to follow the circumference of the port opening. The aperture 406 may generally follow the shape of inner circumference of the port 111, 159. It may extend for a portion, or substantially all of the circumference of the port. It may extend for a segment of up to 360 degrees, up to 180 degrees or up to 90 degrees. It may extend for about 135 degrees, or between 90 and 180 degrees, or between 120 and 150 degrees. The segment may be annular as shown in FIG. 7. In some cases, the width of the tube winder 400 (that is to say the narrower dimension) is substantially the width of the port. The overall width may be less than the width of the port and one, or a plurality of tube windings—as the tube is wound at least once around the tube winder 400. This narrow dimension reduces the space occupied by the tube winder 400, except in the elongate dimension. In some cases, tube holder 410 extends beyond this dimension, but the remainder of the tube winder 400 does not.

FIG. 8 shows the view of the tube winder 400 at the port connection end. As shown the port connection is formed by an engagement surface formed by the inner side wall 425 and the side wall 420. Alternatively, the side wall 420 may be separate from the engagement surface. In some cases, the engagement surface has a slot 440. The slot 440 allows the sides of the engagement surface to resiliently deform so recesses 442 can slide over a corresponding protrusion (such as a rib) or protrusions on the outside surface of the port. The combination of the slot 440 and the recesses 442 provides both orientation of the tube winder 400 and positioning, securing and/or height control by recesses 442. The slot 440 may widen at the mouth, for example with a slope 441, so as to guide a protrusion onto recesses 442. The mouth of the slot 440 may therefore be wider than the body of the slot 440. In this way the slot 440 guides the tube winder 400 into position on the port 111, 159, as the wide mouth can capture a protrusion and, with further downward pressure on the tube winder 400, urge the protrusion into position in the slot 440.

FIG. 9 shows side wall 420 extending from the top surface 407 to bottom surface 408 continuously. As shown in FIG. 31 approximately six windings of the tube 401 may be wound in each (of one or more) layers from top to bottom, or bottom to top, of the side wall 420. In some cases, the side wall 420 may be discontinuous or have discontinuities. In some cases the surface of the side wall 420 may be substantially flat. In some cases the surface may have protrusions, or be shaped, for example to help position the liquid tube 401. The side wall 420 may refer to a single surface or a plurality of surfaces, or a surface formed by a plurality of separate ribs rather than a surface. The side wall 420 provides or is the support on which the tube 401 is wound. The side wall 420 defines the location of inside of the tube 401, when wound against the tube winder body. For example, the side wall 420 may be formed by a series of protrusions, such as ribs, from one or other, or between, the upper and lower surfaces 407, 408. The upper and/or lower surfaces may comprise a frame suitable to support these protrusions. For example, the side wall 420 may be formed by a plurality of ribs descending from an upper frame or rising from a lower frame. The ribs may extend between the upper and lower frame, or if a single frame is present from that frame. In this way the surface and/or side wall is discontinuous, but the ribs extending from or between the frames provide support for the wound tube.

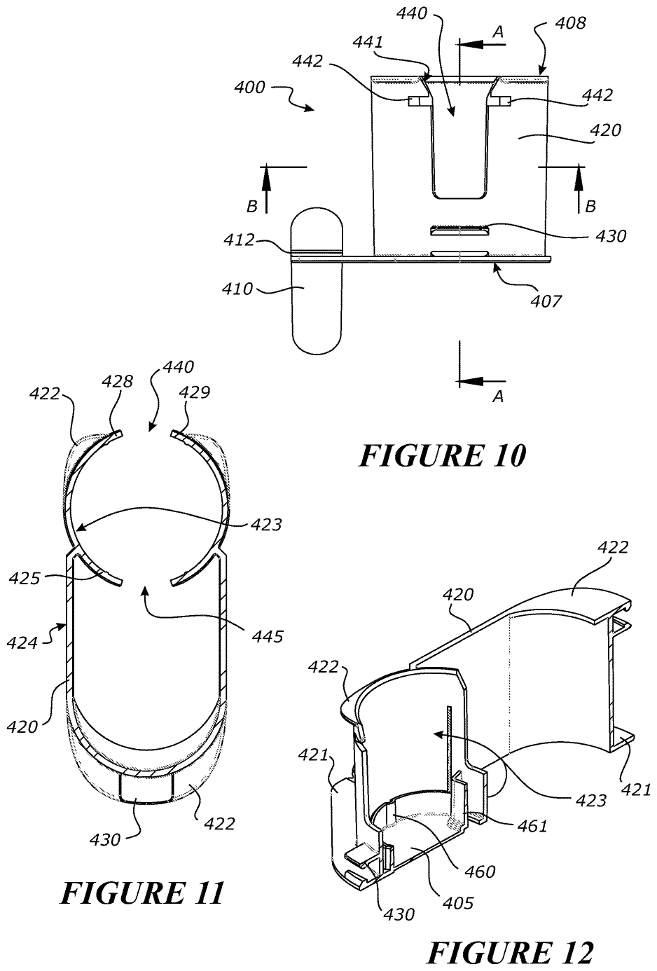

FIG. 10 shows a rear view of the tube winder 400 of FIG. 5 upside-down. FIG. 11 is a horizontal cross section of the tube winder 400 along B-B as shown in FIG. 10 showing the detail of the side wall 420. In the illustrated case, the side wall 420 is formed by a first section at the port connection end, and a second section extending to a distal end to form the elongate body. The first end has two wall portions 428, 429 on either side of slot 440. These portions are resiliently deformable so they can widen or move apart to allow a protrusion(s) through into recesses 442. This also allows the tube winder 400 to be removed from the port, for example by levering it off by upwards force at the distal end. FIG. 11 shows that the engagement surface to the port may be an inner surface 423 of the side wall. FIG. 11 also shows that a second slot 445 may be present on the engagement surface. This increases the flexibility of the engagement surface around the port. In some cases, as shown, the second slot 445 is substantially the same as the first slot 440. In some cases, it is substantially opposite the first slot 440.

FIG. 12 shows a vertical cross section of tube winder 400 along A-A of FIG. 10. This shows additional detail in the engagement surface. The engagement surface may have guides to provide further alignment with the port. For example, recess 460 on an inner wall 461 is configured to mate with a protrusion on the inner wall of the port. The inner wall 461 may be referred to as a locating wall. The recess and protrusion arrangement may be reversed, or an alternative guide may be used. Inner wall 461 may be spaced apart from the engagement surface 423 to allow the port wall to pass therebetween. Inner wall 461 may extend to surround aperture 406, so as to provide additional support and/or orientation to the retainer when being placed or removed from the tube winder 400.

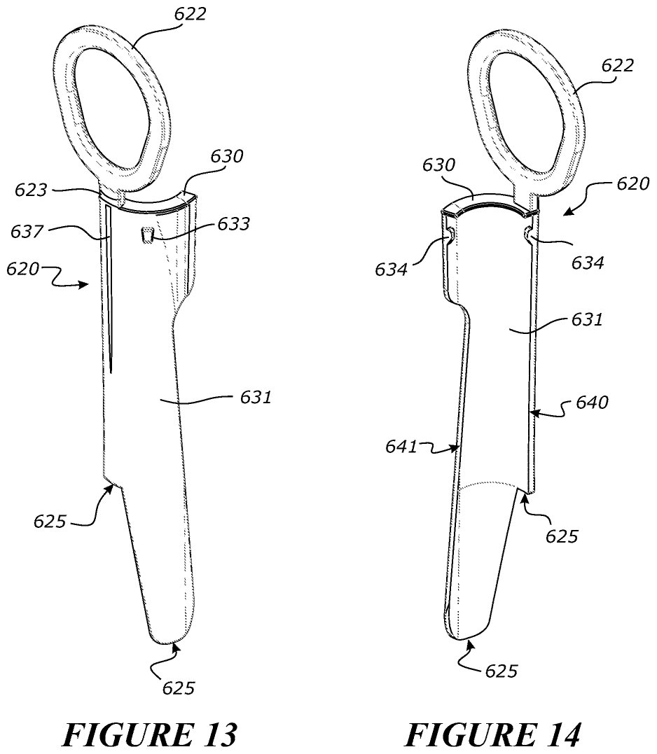

FIGS. 13 and 14 show an example of the retainer. The retainer 620 includes leg 631 configured to extend into the port 111, 159 and/or the humidification chamber 129 and lifting structure such as pull tab 622, which may be formed as a loop. The pull tab 622 advantageously allows the user to remove the retainer 620 during the appropriate stage of set-up. The pull tab 622 is visually intuitive such that the user will typically understand that they are to pull on the pull tab 622 without requiring additional instructions. When engaged with the port of the chamber 129, the retainer 620 extends into the inlet port 159 and/or the outlet port 111 and for example restrains the float(s) 700, 701 in position for shipping. For further discussion of chamber arrangements, including floats and retention thereof, feedsets and example configurations of the broader humidification system, reference is again made to US20170252531A1. The leg is preferably elongate so as to extend for a required distance into the port and/or chamber.

The retainer 620 can include other or alternative orientation and/or alignment features, and or intrinsically provide therefor. In some cases, at least a portion of the retainer 620 is dimensioned so as to fit snugly inside the aperture 406, thereby controlling an alignment of the retainer 620 with respect to the port 111, 159. The retainer 620 may only fit into the aperture in a single orientation. The aperture 406 may be located to urge the retainer 620 against the inner surface of the port, so as to help to locate the retainer 620. The inner wall 461 may also push the leg 631 of the retainer 620 against the inner surface of the port or otherwise help to locate retainer 620. Additionally, or alternatively, the leg 631 of retainer 620 can include one or more projections or recesses that engage one or more corresponding recesses or projections associated with the port 111, 159 (preferably provided on an internal wall of the port) so as to control a rotational orientation of the retainer 620 relative to the port 111, 159 about an axis through the port. For example, in some cases, grooves are configured to receive ribs provided on an inner wall of the port 111, 159.

For example, FIG. 13 shows groove 637 on leg 631 configured to align with a mating projection in the port. The groove 637 may have a taper to help locate the groove relative to the projection in the port when the retainer 620 is inserted into the aperture 406. A projection on the retainer 620 could instead mate with a groove in the port 111, 159 and/or tube winder 400. Projection 633 on the outer surface of leg 631 is configured to engage with a recess in the port to secure the retainer 620 at a particular height, or otherwise align the retainer 620. In some cases, the projection 633 may also or alternatively be located on the inner wall of the tube winder 400. Again, a recess could instead be used. Projections 634 on the edges 640, 641 of the leg 631, shown in FIG. 14 extending radially inwards at the sides of the retainer leg 631 are also used to align the retainer 62. This may be against the inner wall 461 of the tube winder 400 and/or the port 111, 159.

The retainer 620 can be configured to secure one or more floats within the chamber 129 for shipping and/or storage. To this end, the retainer 620 can define or have one or more engagement points 625 on leg 631, each engagement point 625 being configured to engage a respective float and retain the float in a particular position when the port cap assembly is installed on the chamber 129. In some cases, the engagement points retain components other than floats within the humidification chamber 129. Further details of this engagement and the structure of the retainer are provided in US20170252531A1, incorporated herein by reference.

FIGS. 13, 14 and 16 show a retainer 620 comprises a leg 631 with a generally arcuate portion at or near a first or top end which preferably tapers towards the second end by portions of the arc effectively being cut away, narrowed or removed and/or the diameter of the arc reducing towards the second end. As shown, preferably, the arcuate portion is hollow to define a space within the leg 631, and/or reduce the space taken up by the leg 631 in the port. Tapering of leg 631 can aid insertion of the leg 631 through the passageway and into the port of the chamber. Cut-outs and the like perform a similar function but can also assist in enabling proper insertion of the retainer 620 into the chamber 129 as well as providing for the required engagements with the float(s) and/or components.

The chamber 129 may include a baffle that defines a profiled passageway inside the chamber 129. The passageway may be arcuate when viewed from above. In some cases, this arcuate profile is complementary to the profile of the portion of retainer leg 631 that passes therethrough when the tube winder 400 and retainer 620 are engaged with the port 111, 159 of the humidification chamber 129. In some cases, the arcuate profile is complementary to the aperture 406 in the cover 405. By forming the retainer 620 profile to be complementary to the arcuate passageway, the retainer 620 and arcuate wall or passageway can cooperate to control a rotational position of the retainer 620 inside the port and/or the orientation thereof such that the retainer 620 is generally aligned with the port 111, 159. Further, the baffle can assist in preventing liquid in the chamber 129 from spilling as the chamber 129 is transported and control flow of gases into the chamber 129, providing for more effective humidification. This secondary orientation (between the retainer 620 and the chamber 129 or port) can assist or work in conjunction with the orientation between the tube winder 400 and the retainer 620.

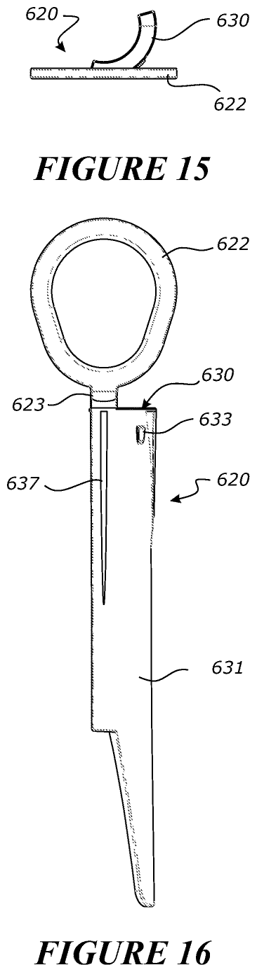

The retainer 620 has an upper surface 630 configured to partially, substantially and/or completely cover the aperture 406 when the retainer is located in the tube winder 400. FIG. 15 shows the upper surface 630 following the arcuate shape of the retainer leg 631 to allow the retainer to be placed vertically (or along the axis of the port) into the port opening. In some cases, the upper surface 630 exactly matches, or substantially matches the shape and/or profile of the aperture 406 so as to form a continuous cover once in place. In some cases, upper surface 630 extends beyond the aperture 406 to create a larger barrier when the retainer 620 is in place.

FIGS. 17, 18 and 19 show the retainer 620 connected to the tube winder 400. As discussed, the retainer 620 passes through aperture 406 in cover 405 configured to sit above the opening of the port 111, 159 and the leg 631 is configured to extend down into the humidification chamber 129. The upper surface 630 of the retainer 620 is shown completely covering the aperture or fitting into a recess at the top of the aperture 406. The shape of leg 631, or the shape of the surface of the leg 631, near the top of the retainer 620 and/or upper surface 630 may be used to orientate the retainer 620 with the tube winder 400. In this way if the tube winder 400 is correctly orientated on the port the retainer 620 will be forced to also be placed in the correct alignment. This may ensure the floats are retained and/or protect the probes, or other components or parts in, or configured to go into, the port and/or humidification chamber 129. FIG. 19 shows that the aperture and/or retainer may be located on the opposite side of the port engagement surface to the slot 440, or at least on the opposite half.

FIGS. 20 and 21 are cutaway views showing detail of the connection between the tube winder 400 and humidification chamber 129. In this case the port is vertically orientated and is an inlet port 159, although other ports may be used. As can be seen an opening 521 for a sensor or probe is located in the port. A grommet 523 may be placed in the opening 521 to allow insertion of the probe(s) and/or sensor(s). Contact between the retainer 620 and the probe in opening 521 could damage the probe and/or the port. For example, by damaging the probe itself and/or the grommet 523, 524. Port 159 is shown with a rib 520 extending horizontally across the outer surface of the port. The rib 520 is configured to engage the recesses 442 on either side of the slot 440. When the tube winder 400 is inserted onto the port the rib 520 will first contact the sloped edges at the mouth of the slot 440. Pressure applied by a user will force the sides 428, 429 (shown in FIG. 11) of the engagement surface apart allowing the tube winder to move further onto the port 159 until it reaches the recesses 442 when it will snap into the position shown in FIGS. 20 and 21. In some cases, protrusions other than a rib 520 may be used. In some cases, separate protrusions may be used on each side of the slot 440 and/or slot 445. In some cases, rib 520 is configured to also mate with a conduit or other item connected to the port, so as to provide a second use of the protrusion. In some cases, the protrusion(s) and recesses may be reversed so that a recess on the port engages a protrusion on the tube winder 400. The rib 520 and slot 440 provide a guide to urge the correct orientation of the tube winder 400. The sloped sides at the mouth of slot 440 guide rib 520 into the correct position. The slot 440 also limits the ability of a user to incorrectly connect the tube winder 400, as it requires one, or more, selected orientations to be used. In some cases the ends of rib 520 may be tapered to guide the position of the tube winder 400.

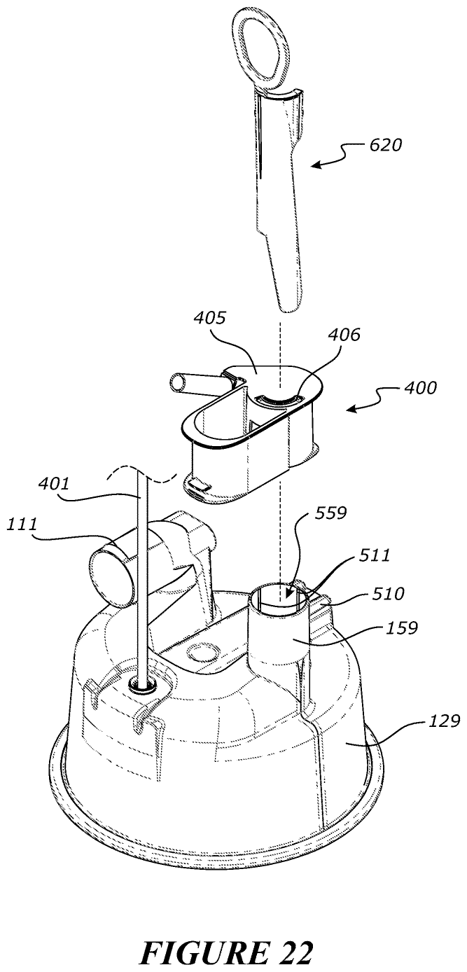

FIG. 22 shows an exploded diagram of the retainer 620, the tube winder 400 and the humidification chamber 129. The dashed line indicates the movement of the retainer 620 and tube winder 400 to secure to the humidification chamber. In use the tube winder 400 will be wound with liquid tube 401, which has a first end attached to the humidification chamber 129. The tube winder 400 is then positioned onto the port. In some cases, this is inlet port 159 or outlet port 111. The connection between the tube winder 400 and the humidification chamber may have an orientator to control the direction of the elongate extension of the tube winder 400. This may include protrusions 511, such as ribs, on the inner surface of the port. The cover 405, that forms part of upper surface 407 of the tube winder 400, is arranged above the opening 559 of the port. Aperture 406 is arranged in cover 405 so as to provide access to the port opening 559. This allows retainer 620 to pass through the aperture and into the humidification chamber 129. In some cases, protrusions 511 are also used to guide or orientate the retainer 620. In one example the protrusions 511 support or engage the edge of the retainer leg 631. A second example, mentioned above, the protrusions 511 engage a recess on the retainer leg 631, such as groove 637 shown in FIG. 13. Although shown as attaching to the vertical port 159 the tube winder could similarly be attached to the horizontal port 111. For example, the elongate extension may then be arranged vertically, or to a side of the port and/or humidification chamber.

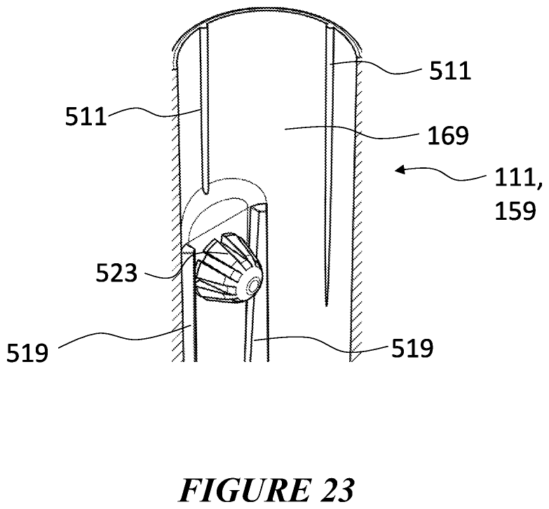

In some cases additional protrusions 519 may be present on the inside wall 169 of the port(s). The protrusions may be axial (along the length of the port) or radial (around the circumference of the port). FIG. 23 shows the inside of a port 111, 159. Although shown as a vertical port the port may be aligned horizontally or in another orientation. The inside wall 169 of the port 111, 159 shows protrusions 511 extending axially. As explained above these protrusions 511 can be used to guide or orientate the retainer 620 when placed in the port 111, 159. In some cases additional protrusions 519 may be included on the inside of the port 111, 159. The additional protrusions may be arranged on the inside wall 169 of the port 111, 159.

FIG. 23 shows protrusions 519 on both sides of the grommet 523. The protrusions 519 may be referred to as protective protrusions, because they protect the grommet 523. In some cases a single protrusion 519 is present. The protrusions 519 are shown adjacent the grommet 523. In some cases the protrusions 519 are spaced apart from the grommet 523. The location of the protrusions, either relative to the grommet 523 or relative to the port 111, 159 may depend on the shape of the port 111, 159 or the leg 631 of retainer 620. FIG. 23 shows protrusions 519 beginning above the grommet 523 and extending into the port 111, 159. In some cases the protrusions 519 extend further towards the opening of the port 111, 159, or to the opening of the port 111, 159. In some cases the protrusion(s) 519 extend to just above the grommet 523. This may be within 5 mm of the edge of the grommet nearest the opening of the port 111, 159. The protrusions 519 may have a constant profile as they extend into the port. However, and as shown in FIG. 23, the protrusions may widen as they extend into the port. The widening may extend towards the grommet 523. This provides a substantially straight edge facing away from the grommet 523. This may also provide additional strength to the protrusion 519 due to more area being in contact with the wall of the port 111, 159. Although not shown in FIG. 23 the protrusions 519 may meet below the grommet 523 in some cases.

The protrusion(s) 519 may provide further protection for the grommet 523 by preventing objects from shearing against and dislodging or damaging the grommet 523 or contacting the side of the grommet 523. In some cases a user may attempt to rotate the tube winder 400 during removal. This rotation could lead to the retainer 620 shearing against the grommet 523. Protrusion 519 helps to prevent the rotation, or at least rotation against the grommet 523. In some cases the side of the protrusion facing away from the grommet 523 may be used to locate an object, such as the retainer leg 631. In use the retainer leg 631 may abut the outside edge of the protrusion. In some cases the retainer leg 631 is further located by protrusions 511 which engage grooves 637 on the leg 631. In some cases the recesses and protrusions may be swapped between objects to similar effect. In some cases the grommet 523 is located in an opening through a thicker section of the wall of the port 111, 159. In this way the thicker wall provides protection and alignment equivalent to protrusions 519, but the grommet 523 may extend further into the port, or at least the base of the grommet 523 is further from the outer wall of the port 111, 159.

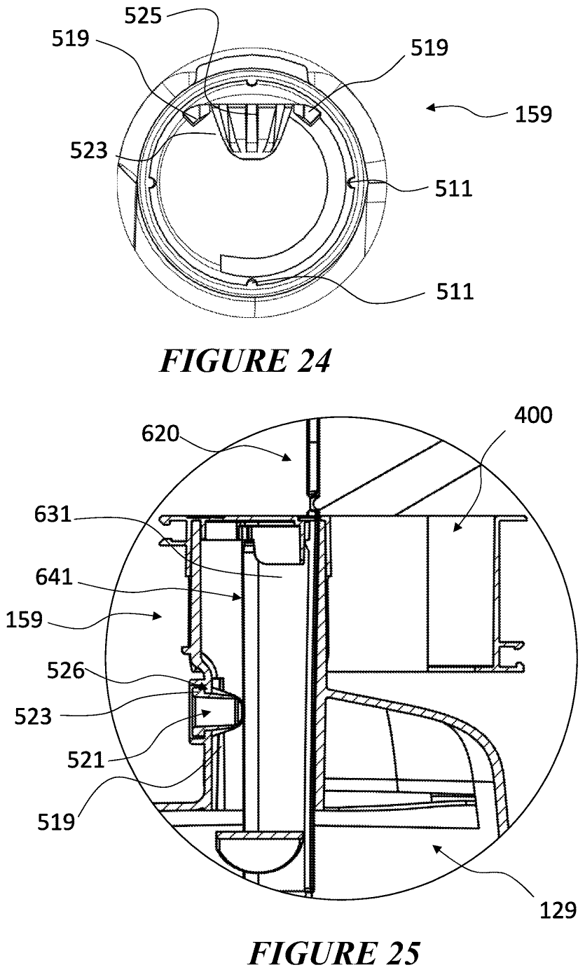

FIG. 24 shows detail of the grommet 523 in the port 111, 159 opening. The grommet 523 is shown as extending through the port 111, 159 to protect and/or shelter a sensor passing through the opening. The grommet 523 has a plurality of ribs 525 meeting at a centre. The ribs 525 provide strength to the grommet 523 in use, preventing the grommet 523 from being pushed against or otherwise contacting a sensor. Alternative forms of grommet 523 may be used to protect the sensor from contact. The contact may include with the port opening 112, 521 or objects within the port.

FIG. 24 shows a plan view of the grommet 523 in a vertical port 159. A similar arrangement may be used with a horizontal port, or as shown in FIG. 31 the horizontal port may still have a horizontal opening. The grommet 523 passes through the side wall of the port 159 to allow a sensor to pass into the port 159. The grommet 523 extends into the port 159. The grommet 523 may extend towards the center of the port 159. The grommet 523 may not extend to the center of the port 159. As described above the grommet 523 form a barrier to the sensor to allow re-use without cleaning. The barrier may be formed by a closure of the grommet. FIG. 24 also shows the protrusions 511 on the inner wall of the port 159. As shown, these may be equally spaced around the inner wall. There may be four protrusions 511, although other amounts may also be used.

FIG. 25 shows a cut-away view of the vertical port 159 showing the of the grommet 523 passing through the wall of the port 159 in opening 521. A portion of the chamber 129, the retainer 620 and the tube winder 400 are also shown. The grommet 523 may be secured to the wall by a recess such as circumferential channel 526 around the grommet. Alternative techniques may be used. It can be seen that the opening 521 in the port 159 is located in a narrower section of the port 159 or on a recess in the port wall. This helps to position the grommet 523, and therefore a sensor within the grommet, closer to the centre of the port 159. It may also provide protection from objects being inserted from above. However the port 159 may have a constant cross-section in some cases. FIG. 25 shows the protrusions 519 may be located on the narrower portion of the port 159. In some cases the protrusions 519 are located on the edges of the narrow portion of port 159.

Protrusion 519 is shown extending lengthwise along the inner wall of the port 159 and protruding towards the centre of the port. The protrusion 519 is located near or next to the grommet 523. In some cases further protrusions 519 may be used. For example a radial protrusion, following the internal circumference of the wall of the port 159 may be positioned above the grommet 523 to provide further protection from above. FIG. 25 shows the leg 631 of the retainer 620 extending into the port 159. In this case the retainer 620 is secured in a position such that the leg 631 is spaced apart from the protrusion 519. However, the edge, for example 641, may be configured to abut the protrusion 519 in some cases. The protrusion continues to provide protection once the tube winder 400 has been removed, particularly if the retainer 620 is reinserted. In some cases the protrusion 519 prevents rotation of the retainer 620 over the grommet 523. Where the retainer leg 631 has a curved edge 641 the protrusion may optionally be curved or shaped to engage the curved leg, or to engage an outermost point on the edge.

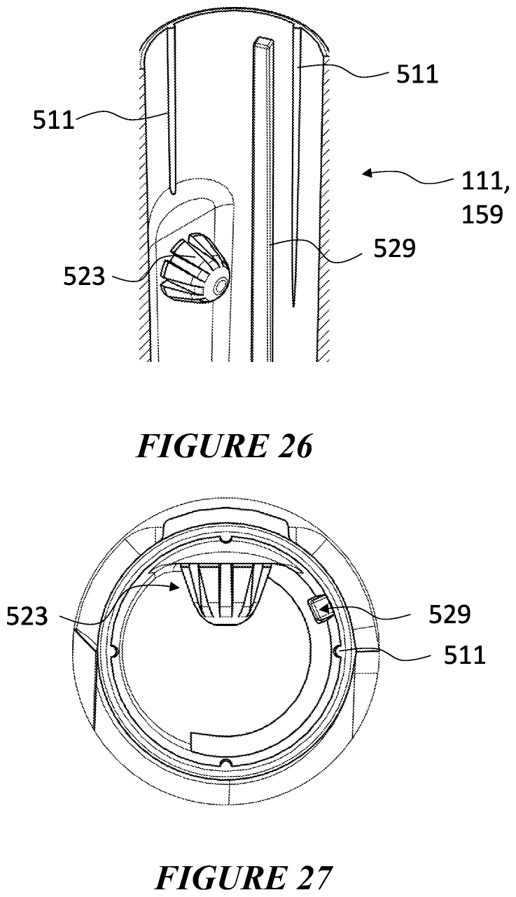

FIG. 26 shows an alternative protrusion 529. The protrusion 529 may have the same features or characteristics as protrusion 519. A single protrusion 529 is shown. The protrusion 529 is a vertical rib. The protrusion 529 is parallel to the axis of the port 111, 159. The protrusion 529 is spaced apart from the grommet 523. The protrusion 529 is separated from the narrowed portion of the port wall through which the grommet 523 passes. The protrusion 529 extends above and below the grommet 523. The protrusion 529 extends substantially above the grommet to the opening of the port 111, 159. However the protrusion is spaced apart from the opening of the port 111, 159. In some cases the protrusion extends to the opening of the port 111, 159. The protrusion 529 has a larger cross-section than the protrusions 511 also in the side wall of the tube. This helps to prevent rotation of the retainer 620 past the protrusion 529.

FIG. 27 shows a plan view of the protrusion 529 and grommet 523 in a vertical port 159. Again, a similar arrangement may be used with a horizontal port, or as shown in FIG. 31 the horizontal port may still have a horizontal opening for the grommet 523. A single protrusion 529 is shown however one or more protrusions 529 may be included. For example a second protrusion 529 may be included on the opposite side of the grommet 523. The larger protrusion 529, when compared to the protrusion 519, provides a more secure stop for the edge 641 of the retainer 620. The protrusion 529 is also moved further from the grommet 523 to abut the leg 631 of the retainer 620 when the retainer is connected to tube winder 400. The protrusion 529 may be spaced by at least 45 degrees around the port from the centre of the grommet.

FIG. 28 shows a cut-away view of the vertical port 159 showing the grommet 523 passing through the wall of the port 159 in opening 521. A portion of the chamber 129, the retainer 620 and the tube winder 400 are also shown. It can be seen that the opening 521 in the port 159 is located in a narrower section of the port 159, or through a recess in the port wall. FIG. 28 shows the protrusions may be located away from the narrower portion of the port 159. This provides a wider protected space around the port. As shown the protrusion 529 extends lengthwise along the inner wall of the port 159 and protrudes towards the central axis of the port. In some cases further protrusions 529 may be present. Both vertical ribs, as shown and/or horizontal ribs may be used. Angled protrusions may also be used. FIG. 28 shows the leg 631 of the retainer 620 extending into the port 159. In this case the retainer is secured in a position such that the leg 631 abuts the protrusion 529. The edge 640, 641 of the retainer 620, for example edge 641 (although either or both edges may be used), is configured to rest against, or at least be stopped by the protrusion 529. The protrusion 529 continues to provide protection once the tube winder 400 has been removed from the port 159, particularly if the retainer 620 is reinserted. In some cases the protrusion 529 prevents rotation of the retainer 620 over the grommet 523. Where the retainer leg 631 has a curved edge 641 the protrusion may optionally be curved or shaped to engage the curved leg, or to engage an outermost point of the edge.

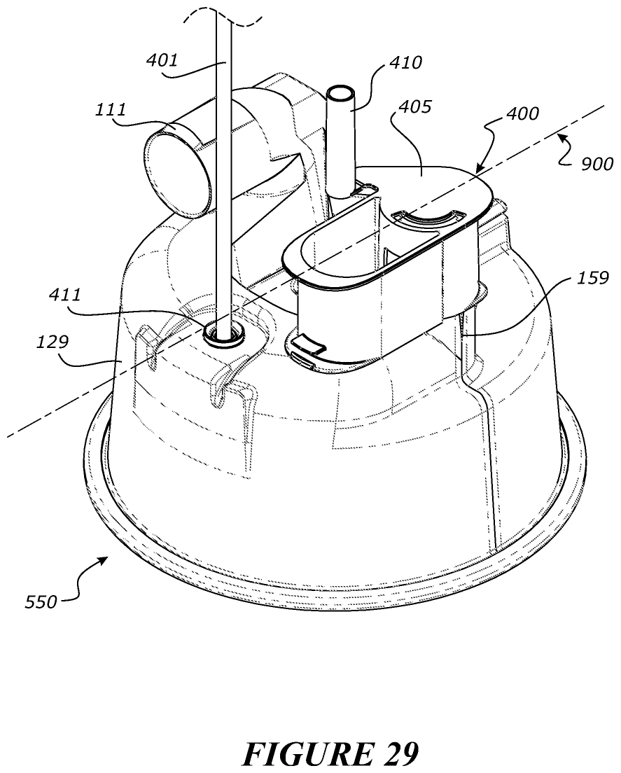

FIG. 29 shows the tube winder 400 mounted on or engaged with the humidification chamber 129. The tube winder 400 varies from the tube winder shown in FIGS. 2 to 9 as the holder 410 has been moved to the opposite side of the upper surface 407 of tube winder 400. Other positions or connections of the holder 410 are possible. The liquid tube 401 is shown extending from the humidification chamber 129 through outlet or connection 411. The tube winder 400 has been orientated so as the elongated direction (following axis 900) extends to towards the front 550 of the humidification chamber. The front 550 of the humidification chamber 129 is substantially opposite the side 551 of the humidification chamber which connects to the humidification base 102. This means that the tube winder 400 extends away from potential connections reducing the likelihood of it getting in the way and/or needing to be removed during set-up and/or installation. In some case the tube winder 400 does not extend directly towards the front. In some cases, the tube winder is offset from axis 900 so as to still extend towards the front but at an offset angle. The humidification chamber 129 may slide onto the humidification base 102 in a first direction, where the front of the humidification base is brought into connection with the back of the humidification chamber (i.e., in a direction up the page in FIG. 4). FIG. 29 shows the tube winder 400 extending in a second direction. In the illustrated example the second direction is opposite, or 180 degrees, from the first direction so as to point towards the front of the humidification chamber (i.e., along axis 900). In some cases, the second direction is at a different angle, for example directed radially outward, or away from the centre of the humidification chamber 129.

FIG. 30 shows a view of FIG. 29 from the back of the humidification chamber. The back 551 of the humidification chamber 129 is the side on which the humidification chamber 129 connects to the humidification base 102. As shown the top surface 560 of the humidification chamber 129 may be configured to engage with the base 102 and/or cartridge 300. A raised portion 510 or groove may engage a corresponding groove or protrusion on the humidification base 102 or cartridge. In some cases, this, or other orientation features, are used to control the orientation of the humidification chamber 129 and the humidification base 102, ensuring that the orientation of the components orientated with respect to the humidification chamber 129 are correctly orientated with respect to the orientation base 102 and vice-versa. Thus, it is advantageous that the tube winder 400 extends away from the raised portion 510 of the humidifier surface. In some cases, one or more openings 521, 112 in the ports 159, 111 are configured. For example, these allow probes to enter the ports. The openings 521, 112 may have seals, such as grommets 523, 524 for protection of the probes, sensors and/or ports. As shown the tube winder 400 may extend away from the openings 521 so as not to cover or prevent access to the openings 521. Again, this allows an easier set-up of the device. Slot 440 and rib 520 are configured to connect the tube winder 400 so that it sits above the opening 521 on port 159. This reduces interference between the tube winder 400 and opening 521 in use. FIG. 30 also shows the base plate 504 and rim 506 of the humidification chamber. These may be configured to improve engagement between the humidifier base and the chamber as described above.

FIG. 31 shows the tube winder 400 engaged to the humidification chamber 129 with liquid tube 401 wound about the side wall of the tube winder 400. A tube retainer is shown as a clip 430 retaining the liquid tube 401 on the tube winder 400. As shown a single thickness of liquid tube 401 is wrapped around the side tube winder 400, although it is possible that two, three or a plurality of layers of liquid tube 401 may be wound about the tube winder 400. Six layers of windings are arranged on the outer surface of tube winder 400. In some cases, between two and ten layers may be wound, or between 3 and 9, between 4 and 8 or between 5 and 7. The retainer 620 is positioned in the tube winder 400, with pull tab 622 extended above to allow release of the retainer 620. The leg 631 extends inside the humidification chamber 129. As shown Figure in 31 retainer 620 makes contact with first and second floats 700, 711. Contact is shown at retaining points 701, 712 on the floats. In some cases, instead of contacting the float 711 directly a retainer, such as a protrusion, extends from the float to provide a suitable contact surface 712 or provide suitable leverage.

FIG. 32 shows the side view of FIG. 31, again showing the components, such as floats 700, 711 within the humidification chamber 129. A first end of the liquid tube 401 is shown connected to the humidification chamber at port 411, with the second end having water spike 402 attached and being stored in the holder 410. The front 550 and opposite back 551 or connection surfaces of the humidification chamber are shown, with the alignment of the elongate extension of the tube winder 400 configured to extend away from the back surface 551. The humidification chamber slidably connects to the humidification base 102 at back surface 551. This side view also shows the grommet 524 in opening 521 extending into the port 159. This shows the importance of orientating the leg 631 of the retainer 620 so as to avoid contact with the grommet 524. The base 128 of the humidification chamber 129 which is configured to engage the heater plate on the heater base. In some cases, the base plate 504 and lip 506 provide engagement to the heater base.

The side wall 420 may have features configured to improve the support or retain the tube 401 on the tube winder 400. In some cases, the outer side wall comprises at least one tube retainer for retaining the wound tube against the outer side wall. In some cases, a lower flange 422 is used to prevent the tube from sliding downwards. In some cases, an upper flange 421 is used to prevent the tube from sling upwards. In some cases, both upper and lower flanges are used. The flanges 421, 422 may be substantially continuous around the circumference of side wall 420 or may be discontinuous. As illustrated the flanges extend on each end 470, 471 of the elongate axis of the side wall 420. In other cases, the flanges may extend on at least two sides of the side wall 420 or tube winder 400.

Tube retainers, such as clips 430 shown in FIGS. 5 and 6 may be used to retain the liquid tube to the side wall 420. Clips 430 are shown as a protrusion which is spaced apart from flange 422 by substantially the width of the tube to be retained. In some cases, the spacing is slightly less than the width of the tube so as to create a retaining force when the tube is placed into the clip 430. For example, the protrusion may deform to allow the tube to enter between the two arms of the clip. In some cases, the clip is formed between a flange 421,422 and a clip arm on the side wall 420. In other cases, the clip 430 is formed by two clip arms on the side wall 420. A second tube retainer, also a clip 430, is shown at the opposite end of the tube winder 400, but between a clip arm and the upper flange. In some cases, the tube winder has first and second tube retainers to retain first and second positions on the tube. In some cases, the tube winder 400 has upper and lower tube retainers so the tube can be easily wound from top to bottom (or from upper surface 407 to lower surface 408) of the tube winder 400. Other tube retainers are possible where they are configured to secure or retain the tube on the tube winder 400. For example, an opening through, or recess in, the side wall 420 may be used. The aperture may have a height substantially equal to (or slightly smaller than) the tube so the tube can be pressed into the recess or through the opening, securing it in place). In some cases, the first and second tube retainers can be different from each other.

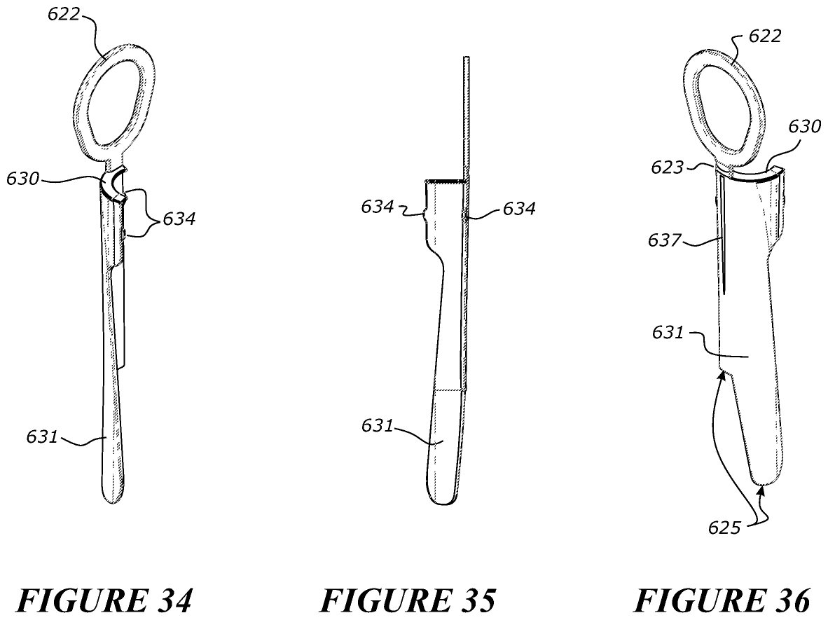

FIGS. 34, 35 and 36 show an alternative arrangement of the retainer 620 shown in FIGS. 13 and 14. It has the pull tab 622, above upper surface 630 and retainer leg 631 configured to extend into the port and/or humidification chamber 129. However, the alignment features are different. While groove 637 remains on the leg 631 the protrusions 634 extend circumferentially at the edges of the leg 631, rather than radially inward as shown in FIG. 14. This enables engagement with different corresponding features in the port and/or tube winder 400. Protrusion 633 shown in FIG. 13 has been removed, although in other cases protrusion 633, or other protrusions on the inner and/or outer surface of the leg 631, are present. Further variations are possible, in particular the shape of leg 631 and the leg edges 640, 641 may be adjusted depending on the particular humidification chamber 129 and/or the components to be retained within.

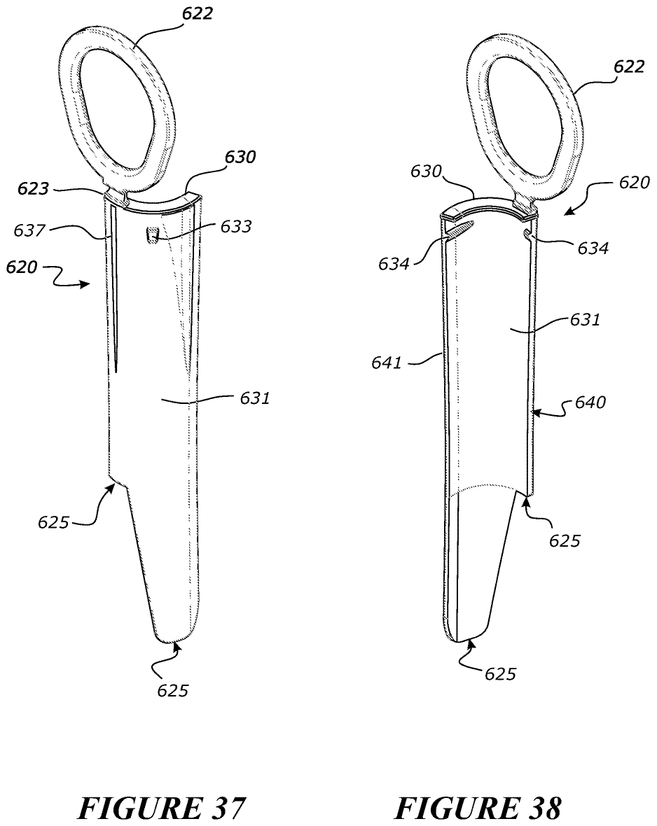

FIGS. 37 and 38 show a further example of retainer 620. The edge 641 of leg 631 is now substantially straight. This means that the width of the leg 631 is substantially constant for a portion of the length of the leg 631. FIGS. 37 and 38 show a constant width of the leg 631 between the upper surface 630 until a first engagement point 625, where a taper begins to a lower engagement point 625. This removes the recess in edge 641 shown in FIG. 14. The second edge 640 is also shown as having a substantially straight portion (or vertical portion when arranged in a vertical port). In some cases, second edge 640 may have one or more recesses or may be shaped to provide a desired engagement point or fit into the port. FIGS. 37 and 38 demonstrate that protrusions 634 may extend along the inner surface of the wall of the retainer 620, as shown by the left protrusion. The extension along the wall may allow the protrusion to be shaped, such as the taper shown, to improve connection to the corresponding feature of the tube winder 400 and/or port. In some cases, both protrusions 634 may extend along the retainer wall. In some cases, a continuous protrusion 634 may extend along the side wall. The protrusions 634 allow the retainer 620 to remain attached to the tube winder 400 unless a force is applied between them. The size and/or shape of the protrusion 634 may be configured to control the amount of force required to remove the retainer 620. Three examples of retainer 620 have been shown, the features of the retainer 620, including the shape of surface 631, the engagement points 625, the shape of edges 640, 640, the shape of projections 633, 634 and the shape of groove 637 may be interchanged between the examples.

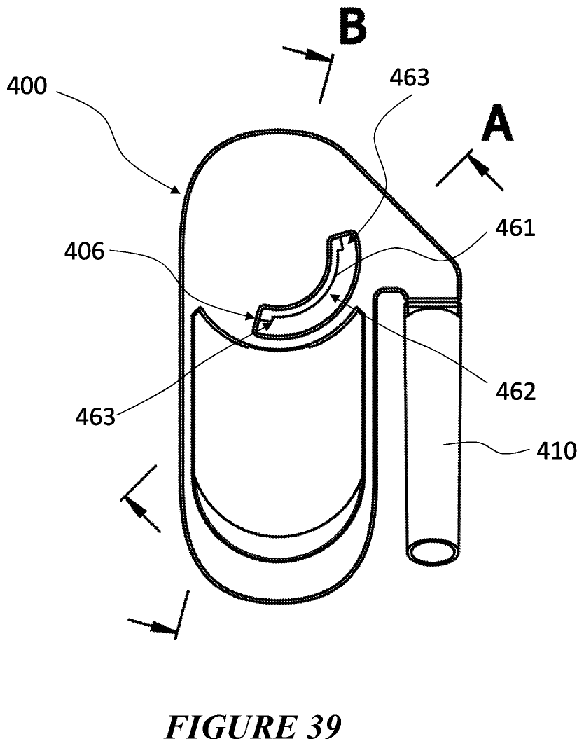

The tube winder 400 may be modified to better connect with the further examples of the retainer 620. For example the tube winder 400 may have additional recesses configured to engage the protrusions 634. FIG. 39 shows an alternative tube winder 400. Compared to FIG. 7 we can see the inner wall 461 (visible through aperture 406) now has a non-uniform profile. The inner wall provides a second engagement surface so the tube winder 400 can secure to both the inner and outer surface of the port wall. The inner wall 461 has a guide portion 462 which is thinner than protrusions 463. The protrusions 463 may be located at the edges of the guide portion 462. The protrusions 463 may form an outer portion of the inner wall. The protrusions 463 may be configured to engage the protrusions 634 of the retainer 620. In use a releasable connection forms between the retainer 620 and the internal wall 461. Although shown as engagement between protrusions 634 and protrusions 463 other releasable connections may be used. For example a recess or opening may be present in the inner wall 461. As shown the protrusions 463 create a recess below them, and into which protrusions 634 may extend. The size and flexibility of the protrusions 634 can be configured to control the force required to remove or insert the retainer 620. Holder 410 is shown on other opposite side of the tube winder 400 compared to the tube winder 400 shown in FIG. 7. Other locations for the holder 410 may also be used.

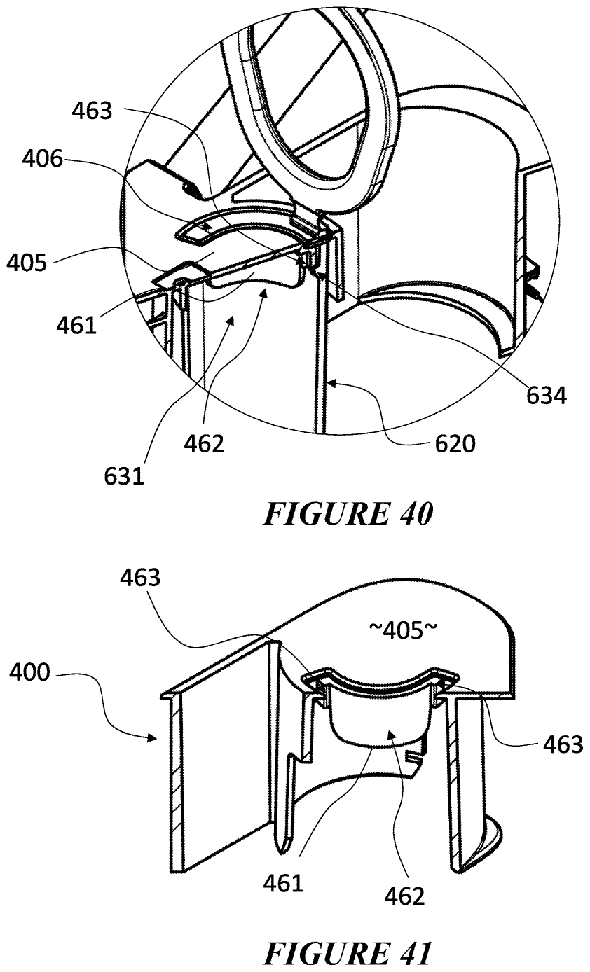

FIG. 40 shows a partial cross-section of tube winder 400 along line B of FIG. 39. Inner wall 461 extends downwards from the cover 405 of the tube winder 400. The inner wall 461 extends furthest where it abuts the leg 631 of the retainer 620. This guides or improves the vertical alignment of the retainer 620 during insertion. As also seen in FIG. 12 the inner wall 461 may extend around the circumference of the cover. In some case the inner wall 461 may instead only encircle the aperture 406. FIG. 40 shows the protrusion 634 of retainer 620 fitting underneath protrusion 463 if of the inner wall. The protrusion 463 is shorter than the adjacent inner wall 461, guide portion 462. This provides the recess for the protrusion 634 to connect to. In some cases the recess may be instead formed by a hole or opening in the inner wall 461.

FIG. 41 shows a cross-section of tube winder 400 along line A of FIG. 39. Again the guide portion 462 is shown extending downwards from cover 405 on the inner side of the aperture 406. Protrusions 463 are located on either side of the guide portion 462 of the inner wall 461 to connect to the retainer 620. The inner wall is shown as being approximately twice as deep as the projections 463, however it may extend further downwards. In some cases it is the same length as the projections 463. However, this may not locate the retainer 620 as effectively as it could rotate about the inner wall 461. The guide portion 462 is shown as being offset from projections 463. This improves the connection with the retainer 620. However, in some case the surface of the guide portion 462 may be continuous with the projections 463, so as to have a constant radius of the inner wall 461.

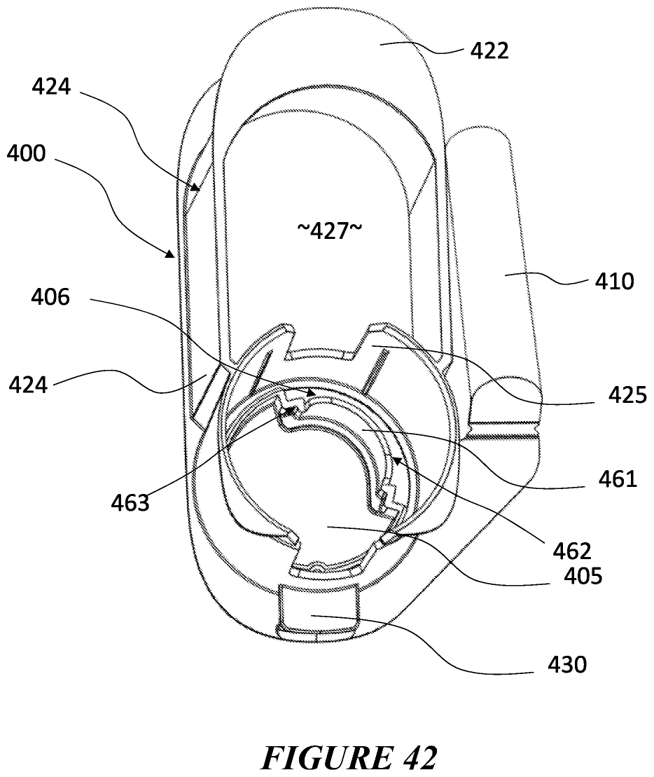

FIG. 42 shows a perspective view of the tube winder of FIG. 39 from below. This provides a view of inner wall 461. Inner wall 461 extends down from the cover 405. As shown the inner wall 461 may extend 360 degrees around the tube winder. This creates a gap between the inner wall 461 and side wall 420 into which the port 111, 159 can slide, securing the tube winder 400 into place. In an alternative embodiment the inner wall 461 may only extend for a portion of the circumference. For example, it may only extend about the aperture 406. The inner wall 461 has a guide portion 462 at the aperture 406. This helps to align the retainer 620 along the axis of the port. It will be understood the the inner wall 461 can provide a stop for the retainer 620. In one example, where the retainer 620 has a protrusion 633 on its outer surface which pushes the retainer leg 631 into contact with the inner wall 461. In conjunction with the protrusions 634, which prevent rotation or further movement of the retainer 620, a 3-point bending mechanism may be provided which locks the retainer in position. The locked position may be overcome by an upwards force applied to the retainer pull tab.

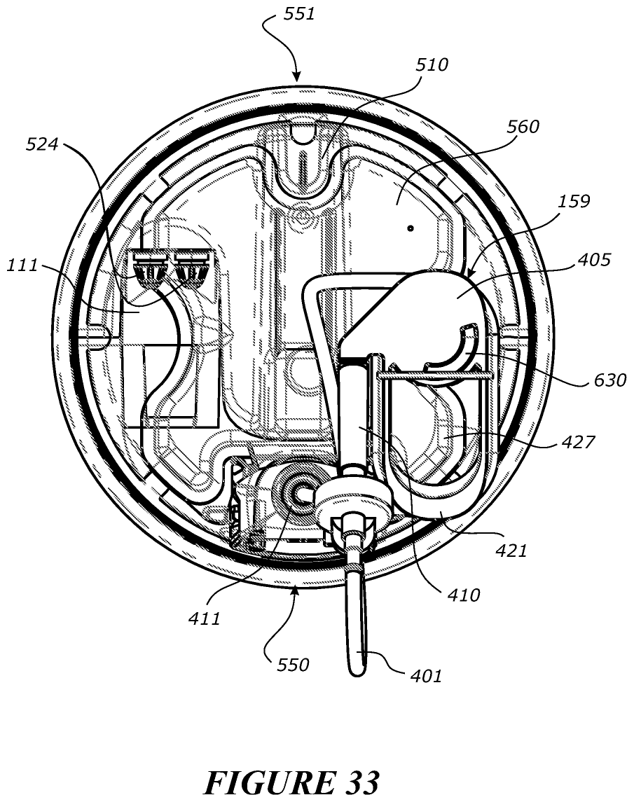

The plan view shown in FIG. 33 shows the structure and dimensions of the tube winder 400 may be configured to reduce overlap of the tube winder and the connection or back side or surface 551 of the humidification chamber 129. As shown, the tube winder 400 or at least side wall 420, is substantially the width of port 159 and while it surrounds port 159 it extends only away from the back side 551. Or at least the outer wall to support the tube only extends in one direction away from the port and/or engagement portion. It may extend directly to the front 550 or may extend towards the front 550 at a selected angle. As shown, the tube winder may also avoid or extend away from a centre of the humidification chamber 129, for example avoiding the area above raised portion 510. This enables the orientation or other features on the top surface 560 of the humidification chamber 129 to be avoided. As illustrated the extension may be substantially parallel to a second port, such as outlet port 111. The body space 427 in the elongate portion of the tube winder 400 may provide vision through to the humidification chamber and/or a suitable holding or leverage point for a user.

The above described examples of a tube winder may control an orientation of the retainer upon insertion not only help to protect against damage to the grommet and/or sensor by reinsertion but also during initial assembly prior to shipment.

As will be apparent to those skilled in the art, such apparatus is ordinarily originally supplied in sealed packaging. To avoid damage to that packaging, in particular puncturing thereof, preferably, the tube winder assembly is configured to avoid sharp edges and protrusions when assembled to a chamber for shipment. For example, rounded corners and edges may be provided on any external edges or corners of the assembly such as around the holder and/or the gripping portion and/or protruding elements may be configured to be flexible.

It should be understood that any examples used in this description are in no way limiting, but merely illustrative of possible examples for purposes of clarification. Unless the context clearly requires otherwise, throughout this description and the claims that follow, the words “comprise”, “comprising”, and the like, are to be construed in an inclusive sense as opposed to an exclusive or exhaustive sense, that is to say, in the sense of “including, but not limited to”.

Reference to any prior art in this description is not, and should not be taken as, an acknowledgement or any form of suggestion that the prior art referenced forms part of the common general knowledge in any relevant field of endeavour in any country in the world.

The present invention may be said broadly to consist in the parts, elements, and features referred to or indicated in this description and the claims that follow, individually or collectively, in any or all combinations of two or more of said parts, elements, or features. Where reference is made to integers or components having known equivalents thereof, those equivalents are herein incorporated as if individually set forth.

It should be noted that various modifications to the examples disclosed herein will be apparent to those skilled in the art. Such modifications may be made without departing from the spirit and scope of the present invention and without diminishing its attendant advantages. For instance, various components may be repositioned or reshaped as desired. It is therefore intended that such modifications be included within the scope of the invention. Moreover, not all of the features, aspects, and advantages disclosed herein are necessarily required to practice the present invention. Accordingly, the scope of the present invention is intended to be defined only by the claims that follow.

What is claimed is:

1. A tube winder, for a humidification chamber comprising at least one port, the tube winder comprising:
a body configured to connect to a single port of the humidification chamber, the body comprising an outer side wall substantially surrounding the single port, an outer surface of the outer side wall configured to support a wound tube.

2. A tube winder as claimed in claim 1 wherein the body comprises an engagement surface configured to engage the single port of the humidification chamber.

3. A tube winder as claimed in claim 2 wherein at least a portion of the engagement surface extends away from the outer side wall.

4. A tube winder as claimed in claims 2 or 3 wherein at least a portion of the engagement surface comprises at least a portion of an inner surface of the outer side wall.

5. A tube winder as claimed in claims 2 or 4 wherein the body comprises an inner side wall, at least a portion of the engagement surface being on at least a portion of the inner side wall.

6. A tube winder as claimed in claims 2 to 5 wherein the engagement surface is configured to substantially surround the single port.

7. A tube winder as claimed in claims 2 to 6 wherein the engagement surface has a guide configured to orientate the tube winder on the outlet.

8. A tube winder as claimed in claim 7 wherein the guide comprises one or more protrusions and/or recesses configured to mate with corresponding recesses and/or protrusions on the single port to secure and/or orientate the tube winder when the tube winder is connected and/or connecting to the single port.

9. A tube winder as claimed in claims 7 or 8 wherein the guide is resiliently deformable so as the tube winder releasably secures to the port.

10. A tube winder as claimed in claims 7 to 9 wherein the guide comprises a slot extending through the engagement surface and/or the side wall.

11. A tube winder as claimed in claim 10 wherein the mouth of the slot is wider than a body of the slot, so as to guide the tube winder into a correct orientation.

12. A tube winder as claimed in claims 10 or 11 wherein the slot comprises a catch to secure the tube winder on the port.

13. A tube winder as claimed in claims 1 to 12 wherein the body comprises a cover, the cover configured to at least partially occlude the single port when the tube winder is connected to the single port.

14. A tube winder as claimed in claim 13 wherein the cover comprises an aperture configured to be located above the opening of the port when the tube winder is connected to the port, the aperture configured to allow a retainer to pass therethrough.

15. A tube winder as claimed in claim 14 wherein the aperture is configured to locate the retainer apart from a component within the single port when the retainer is located in the aperture and the tube winder is engaged to the single port.

16. A tube winder as claimed in claims 1 to 15 wherein the tube winder body comprises an upper frame and/or a lower frame, wherein the outer side wall extends from or between the upper and/or lower frame.

17. A tube winder as claimed in claim 16 wherein the side wall comprises a plurality of members extending from or between the upper and/or lower frame.

18. A tube winder as claimed in claims 1 to 17 wherein the outer side wall comprises an upper and/or a lower flange extending therefrom, the upper and/or lower flange configured to retain the tube on the outer side wall.

19. A tube winder as claimed in claims 1 to 18 wherein the outer side wall is discontinuous and/or comprises recesses and/or comprises protrusions.

20. A tube winder as claimed in claims 1 to 19 wherein the humidification chamber comprises a second port, wherein the tube winder does not surround and/or contact the second port.

21. A tube winder as claimed in claims 1 to 20 wherein the body is elongate.

22. A tube winder as claimed in claim 21 wherein the body and/or the outer side wall extends in a first dimension substantially the width of the single outlet and extends in a second dimension a length greater than the width of the single outlet.

23. A tube winder as claimed in claims 1 to 22 wherein the outer side wall is substantially rectangular.

24. A tube winder as claimed in claims 1 to 23 wherein the outer side wall comprises curved or chamfered corners.

25. A tube winder as claimed in claims 1 to 24 wherein at least a part of at least one end of the outer side wall is flat.

26. A tube winder as claimed in claims 1 to 25 wherein the outer side wall is configured to retain the wound tube.

27. A tube winder as claimed in claim 26 wherein the outer side wall comprises at least one tube retainer for retaining the wound tube against the outer side wall.

28. A tube winder as claimed in claim 27 wherein the tube retainer comprises at least one protrusion from the outer side wall and/or at least one recess or opening in the outer side wall.

29. A tube winder as claimed in claims 1 to 28 comprising a holder configured to support an end of the tube.

30. A tube winder as claimed in claim 29 wherein the holder extends from, or is connected to, the tube winder body.

31. A tube winder as claimed in claims 28 or 29 wherein the holder is configured to support a spike attached to the end of the tube.

32. A tube winder as claimed in claims 28 to 31 wherein the holder comprises a sleeve configured to hold the tube and/or spike.

33. A tube winder assembly comprising:
a tube winder as claimed in claims 1 to 32;
and a retainer comprising a leg configured to pass through the tube winder when connected to the single port.

34. A tube winder assembly as claimed in claim 33 wherein the leg is elongate and comprises at least one engagement portion configured to engage a surface within the humidification chamber.

35. A tube winder assembly as claimed in claim 34 wherein the engagement portion is at or near an end of the leg.

36. A tube winder assembly as claimed in claim 34 or 35 wherein the surface is a surface of a float and/or float assembly within the humidification chamber.

37. A tube winder assembly as claimed in claims 33 to 36 wherein the tube winder comprises an aperture, the aperture configured to align the retainer relative to the aperture and/or the port.

38. A tube winder assembly as claimed in claim 37 wherein the retainer comprises an aligner and/or an alignment surface configured to align the retainer relative to the aperture and/or the port.

39. A tube winder as claimed in claim 38 wherein the aligner comprises a shape of the retainer and a corresponding shape of the aperture and/or the port.

40. A tube winder as claimed in claim 39 wherein the aligner comprises a shape of the leg.

41. A tube winder as claimed in claim 38 or 41 wherein the aligner comprises at least one edge of the leg and/or an upper surface of the retainer.

42. A tube winder assembly as claimed in claims 38 to 41 wherein the aperture and/or the aligner are configured and/or shaped to allow the retainer to be inserted into the tube winder in a single alignment.

43. A tube winder assembly as claimed in claims 33 to 42 comprising a tube, the tube configured to be wound about the tube winder.

44. A tube winder assembly as claimed in claim 43 wherein the tube comprises a first end connected or connectable to the humidification chamber and/or comprises a second end connected or connectable to a spike.

45. A tube winder assembly as claimed in claim 44 wherein the tube is a liquid tube.

46. A humidification assembly comprising a tube winder as claimed in claims 1 to 45 and a humidification chamber, the humidification chamber comprising at least one port.

47. A humidification assembly comprising a tube winder and a humidification chamber, the humidification chamber comprising:
at least one port,
the tube winder comprising:
a body configured to connect to a single port of the humidification chamber, the body comprising an outer side wall substantially surrounding the single port, an outer surface of the outer side wall configured to support a wound tube.

48. A humidification assembly as claimed in claims 46 or 47 comprising a tube connected to the humidification chamber, the tube configured to be wound about the tube winder.

49. A humidification assembly as claimed in claims 46 to 48 comprising a humidification base, the humidification chamber configured to connect to the humidification base.

50. A humidification assembly as claimed in claim 49 wherein the humidification base comprises a cartridge configured to connected to the humidification chamber.

51. A humidification assembly as claimed in claims 49 or 50 wherein the humidification base and/or cartridge has one or more probes and/or sensors extending therefrom, at least one of the one or more probes and/or sensors configured to engage the at least one port of the humidification chamber.

52. A humidification assembly as claimed in claim 51 wherein at least one of the one or more probes or sensors is configured to extend into a port of the humidification chamber.

53. A humidification assembly as claimed in claims 49 to 52 wherein at least one of the ports has one or more grommets configured to cover an opening into the port to protect an associated probe and/or sensor and/or engage with an associated probe and/or sensor.

54. A humidification assembly as claimed in claim 53 wherein the grommets extend within the at least one port.

55. A humidification chamber as claimed in claims 49 to 52 wherein at least one of the ports is an inlet port and/or and outlet port.

56. A humidification assembly as claimed in claims 49 to 55 wherein the humidification base and/or the cartridge is configured to engage with an upper surface of the humidification chamber to align the humidification chamber with the humidification base.

57. A humidification assembly as claimed in claim 56 wherein the humidification base and/or the cartridge has a protrusion configured to engage the upper surface.

58. A humidification assembly as claimed in claims 49 to 57 wherein the humidification base and the humidification chamber are configured to have one or more electrical, mechanical, and/or pneumatic connections therebetween.

59. A humidification assembly as claimed in claim 58 wherein one of the one or more electrical, mechanical and/or pneumatic connections is provided as part of a connector to an outlet of the humidification chamber.

60. A humidification assembly as claimed in claim 46 to 59 wherein the humidification chamber comprises one or more components within the chamber, the components being configured to be retained.

61. A humidification assembly as claimed in claim 60 wherein one of the one or more components comprises a float configured to control the water level in the humidification chamber in use.

62. A tube winder, for a humidification chamber comprising at least one port and configured to connect to a humidifier base in a first direction, the tube winder comprising:
a body configured to connect to a port of the humidification chamber the body comprising:
an outer side wall surrounding a port, the outer side wall comprising an outer surface configured to support a wound tube, wherein the body, when connected to the port, is configured to extend away from the port in a second direction, the second direction being different to the first direction.

63. The tube winder as claimed in claim 62 wherein the body is configured to only extend away from the port in a second direction.

64. The tube winder as claimed in claims 62 or 63 wherein the outer side wall surrounds a single port of the humidification chamber.

65. A tube winder as claimed in claim 62 to 64, wherein the humidification chamber is connectable to the humidification base while the tube winder is connected to the humidification chamber.

66. A tube winder as claimed in claim 62 to 65 wherein the first direction is substantially opposite the second direction.

67. A tube winder as claimed in claims 62 to 66 wherein the second direction is towards the front of the humidification chamber.

68. A tube winder as claimed in claims 62 to 67 wherein the second direction is substantially parallel to a direction of a second port of the humidification chamber.

69. A tube winder as claimed in claim 62 to 68 comprising an orientator configured to orientate the body relative to the port to control the second direction relative to the first direction.

70. A tube winder as claimed in claim 69 wherein the orientator is configured to orientate the body on the port towards the second direction.

71. A tube winder as claimed in claims 69 or 70 wherein the orientator comprises a protrusion or recess on an engagement surface of the body.

72. A tube winder as claimed in claim 69 to 71 wherein orientator comprises a catch configured to engage with the port in the second direction.

73. A tube winder as claimed in claims 69 to 72 wherein the orientator is part of, or on, the outer side wall of the body.

74. A tube winder as claimed in claims 69 to 73 wherein the orientator comprises a slot on the tube winder body.

75. A tube winder as claimed in claims 62 to 74 comprising a cover to at least partially occlude the port.

76. A tube winder as claimed in claims 62 to 75, wherein the tube winder is as claimed in claims 1 to 46.

77. A humidification assembly comprising:
a tube winder configured to support a wound tube, and
a humidification chamber configured to connect to a humidification base, the humidification chamber comprising:
at least one port,
the tube winder connected to a port of the humidification chamber, wherein the tube winder is configured to remain connected to the humidification chamber while the humidification chamber is connected to the humidification base.

78. A humidification assembly as claimed in claim 77 comprising a tube connected to the humidification chamber, the tube configured to be wound about the tube winder.

79. A humidification assembly as claimed in claims 77 or 78 comprising the humidification base.

80. A humidification assembly as claimed in claim 79 wherein the humidification base comprises a cartridge configured to connected to the humidification chamber.

81. A humidification assembly as claimed in claims 79 or 80 wherein the humidification base and/or cartridge has one or more probes and/or sensors extending from the base and/or cartridge, at least one of the one or more probes and/or sensors configured to engage the at least one port of the humidification chamber on which the tube winder is attached.

82. A humidification assembly as claimed in claim 81 wherein at least one of the one or more probes and/or sensors is configured to extend into one of the at least one ports of the humidification chamber.

83. A humidification assembly as claimed in claims 79 to 82 wherein at least one of the at least one ports has one or more grommets configured to surround an opening into the port and/or engage with an associated probe and/or sensor.

84. A humidification assembly as claimed in claim 83 wherein at least one of the grommets extend within the at least one port.

85. A humidification chamber as claimed in claims 79 to 84 wherein at least one of the ports is an inlet and/or and outlet port.

86. A humidification chamber as claimed in claim 84 wherein the tube winder is configured to connect to the port above the one or more grommet.

87. A humidification assembly as claimed in claims 79 to 86 wherein the humidification base and/or the cartridge is configured to engage with an upper surface of the humidification chamber to align the humidification chamber with the humidification base.

88. A humidification assembly as claimed in claim 87 wherein the humidification base and/or the cartridge has a protrusion configured to engage the upper surface.

89. A humidification assembly as claimed in claims 79 to 88 wherein the humidification base and the humidification chamber are configured to have one or more electrical, mechanical and/or pneumatic connections therebetween.

90. A humidification assembly as claimed in claim 89 wherein one of the one or more electrical and/or pneumatic connections is provided as part of a connector to an outlet port of the humidification chamber.

91. A humidification assembly as claimed in claim 77 to 90 wherein the humidification chamber comprises one or more components within the chamber, the components being configured to be retained.

92. A humidification assembly as claimed in claim 91 wherein one of the one or more components comprise a float configured to control the water level in the humidification chamber in use.

93. A humidification assembly as claimed in claims 77 to 92 further comprising a retainer configured to extend through the tube winder into the port and/or the humidification outlet.

94. A humidification assembly as claimed in claim 93 wherein the retainer is configured to be aligned by the tube winder and/or the port so as to avoid contact with components within the humidification chamber and/or the port.

95. A humidification assembly as claimed in claim 94 wherein the retainer is aligned to avoid contact with a grommet in the port.

96. A humidification chamber as claimed in claims 77 to 95 wherein the tube winder comprises an outer side wall with an outer surface configured to support the wound tube.

97. A humidification chamber as claimed in claims 77 to 96 wherein the tube winder is a tube winder as claimed in claims 1 to 46 or 62 to 76.

98. A tube winder as claimed in claims 77 to 97 wherein the body is configured to connect to a single port of the humidification chamber.

99. A tube winder, for a humidification chamber comprising at least one port, the humidification chamber configured to connect to a humidifier base in a first direction, the tube winder comprising:
a body configured to connect to a port of the humidification chamber,
wherein the body comprises a cover configured to at least partially occlude the opening of the port when the tube winder is connected to the port, the cover comprising an aperture configured to allow a retainer to pass therethrough into the port.

100. A tube winder as claimed in claim 99 comprising an orientator configured to orientate the body relative to the port on the humidification chamber.

101. A tube winder as claimed in claim 100 wherein the orientator comprises a slot.

102. A tube winder as claimed in claim 99 to 101 wherein the aperture is configured to locate a leg of the retainer relative to the port and/or the tube winder.

103. A tube winder as claimed in claim 102 wherein the aperture is shaped to position and/or guide the leg of the retainer relative to the port and/or the tube winder.

104. A tube winder as claimed in claims 99 to 103 wherein the aperture is configured to secure the retainer in a selected position.

105. A tube winder as claimed in claims 99 to 104 wherein the aperture comprises a wall configured to guide the retainer.

106. A tube winder as claimed in claim 105 wherein the wall extends along at least a portion the circumference of the aperture.

107. A tube winder as claimed in claim 106 wherein the wall extends below the cover of the tube winder.

108. A tube winder as claimed in claims 99 to 108 wherein the body is configured to connect to a single port of the humidification chamber.

109. A tube winder as claimed in claim 99 to 108 wherein the cover comprises a closure configured to close the aperture when the retainer is not present.

110. A tube winder as claimed in claims 99 to 109 wherein the cover and the retainer are configured to fully occlude the opening when the tube winder assembly is connected to the port.

111. A tube winder as claimed in claims 99 to 110 wherein the body comprises an outer side wall substantially surrounding the single port, an outer surface of the outer side wall configured to support a wound tube.

112. A tube winder as claimed in claim 111 wherein the outer side wall extends around the cover.

113. A tube winder as claimed in claim 99 to 112 wherein body has an engagement surface configured to engage the single port of the humidification chamber.

114. A tube winder as claimed in claims 99 to 113 wherein the tube winder is as claimed in claims 1 to 76.

115. A tube winder assembly comprising: the tube winder of claims 99 to 114 and the retainer, wherein the retainer is configured to engage a surface and/or component inside the humidification chamber.

116. A tube winder assembly as claimed in claims 99 to 115 wherein the cover and the retainer are configured to fully occlude the opening when the tube winder assembly is connected to the port.
